(19) United States
(12) Patent Application Publication
(10) Pub. No.: US 2018/0136227 A1
(43) Pub. Date: May 17, 2018

(54) Treatment and Diagnosis of Hereditary Xerocytosis

(71) Applicants: Universite d'aix Marseille, Marseille (FR); Assistance Publique-Hopitaux de Marseille, Marseille (FR); Institut National de la Sante et de la Recherche Medicale (INSERM), Paris (FR); Centre National de la Recherche Scientifique, Paris (FR); Universite de Nice Sophia Antipolis, Nice (FR)

(72) Inventors: Catherine Badens, Marseille (FR); Isabelle Thuret, Allauch (FR); Hélène Guizouarn, Nice (FR)

(21) Appl. No.: 15/578,982

(22) PCT Filed: Jun. 15, 2016
(86) PCT No.: PCT/EP2016/063745

§ 371 Date: Dec. 1, 2017

(30) Foreign Application Priority Data
Jun. 15, 2015 (EP) 15305921.7

(51) Int. Cl. G01N 33/68; A61P 7/00; C12Q 1/6883; A61K 31/165; G01N 33/50

(52) U.S. Cl. CPC G01N 33/6872; A61P 7/00; C12Q 1/6883; A61K 31/165; C12Q 2600/118; G01N 2800/50; G01N 2333/705; G01N 2800/22; C12Q 2600/156; G01N 33/502

(57) ABSTRACT

The invention relates to an in vitro method of diagnosis of hereditary xerocytosis in a subject, comprising genotyping the KCNN4 gene encoding the Gardos channel in said subject. The invention also relates to an inhibitor of the KCNN4 protein for use in the treatment of hereditary xerocytosis, in particular in a human subject who is a carrier of the missense mutation c.1055G>A or c.844G>A in the KCNN4 gene.

The present invention relates to the diagnosis of hereditary xerocytosis and the treatment of this disorder.

Water and solute homeostasis is essential for the maintenance of erythrocyte integrity and is controlled via the regulation of monovalent cation content. Several primary disorders of erythrocytes hydration exist and are characterized by an abnormal permeability of the erythrocyte membrane to sodium and potassium, resulting either in swelling or shrinkage of red cells (Rinehart et al., 2010). Clinically, these inherited disorders are associated with chronic hemolytic anemia and are due to defects in various transmembrane ion channels or transporters (Da Costa L, et al., 2013).

Hereditary Xerocytosis, (HX) ([OMIM] 194380), is an autosomal dominant congenital hemolytic anemia characterized by primary erythrocyte dehydration (Miller et al., 1971). In HX patients, red blood cells exhibit an altered intracellular cation content and cellular dehydration which is responsible for an increased erythrocyte mean corpuscular hemoglobin concentration (MCHC) and decreased erythrocyte osmotic fragility (Archer et al., 2014). Under the microscope, blood films show various shape abnormalities, the most characteristic being a central pallor, straight or crescent-shaped, which leads to the denomination of stomatocyte for these cells and of Dehydrated Hereditary Stomatocytosis as an alternative name for HX (Da Costa et al., 2013).

HX has been associated with missense mutations in FAM38A encoding the red cell membrane mechanosensitive cation channel, PIEZO1 (Zarychanski et al., 2012; Andolfo et al., 2013a). Functional studies have demonstrated that in PIEZO1, the mutations slowed channel inactivation and introduced a pronounced latency for activation (Bae et al., 2013). More recently, another type of red cell ion exchange defect associated with pseudohyperkalemia has been linked to mutations in the ATP binding cassette transporter ABCB6 (Andolfo et al., 2013b). Rinehart et al. (2010) report that a locus for hereditary xerocytosis has been mapped to 16q23-q24, but the affected gene has not yet been identified.

The Gardos channel is a cation channel also referred to as KCa3.1 or KCNN4. It is a Ca²⁺ sensitive, intermediate conductance, potassium selective channel, initially described in pancreas cells but present in many cell types including erythrocytes (Maher and Kuchel, 2003). The locus of the gene encoding the Gardos channel (KCNN4 protein) is mapped 1903.2. The Gardos channel is made of 4 identical subunits; each subunit is encoded by a single gene, KCNN4, and comprises 6 transmembrane domains and a pore region between the 5^{th} and the 6^{th} transmembrane domains (Maher and Kuchel, 2003). In steady state conditions, the Gardos channel is inactive. Its function is not fully elucidated in mature normal erythrocytes. Under external stimulation, intracellular Ca²⁺ increases and then interacts with Calmodulin molecules that are bound tightly on each of the four channel subunits of the Gardos channel. Ca²⁺ binding to Calmodulin results in the opening of the channel and rapid K⁺ and water efflux leading to erythrocyte dehydration and shrinkage, a mechanism referred to as the Gardos effect (Maher and Kuchel, 2003; Fanger et al., 1999). Red blood cells are in constant movement during blood circulation where they experience mechanical stress on their membrane. Using on-cell patch clamp experiments, it has been shown that local membrane deformation can act as a stimulating event in red cells and lead to Gardos activation, suggesting that this mechanosensory mechanism may allow erythrocytes to adapt their volume and shape to pass through the narrow capillaries of the microvasculature (Dyrda et at, 2010). A number of recent studies have described its role in a variety of physiological events and pointed it out as an interesting therapeutic target in a large panel of human diseases (Wulff and Köhler, 2013; Wulff and Castle, 2010).

The inventors have identified a missense mutation (p.Arg352His mutation) located in one of the functional regions of the Gardos channel and its association with chronic hemolysis and dehydrated cells in two unrelated HX families with eight affected HX persons. The affected individuals present chronic anemia that varies in severity. Their red cells exhibit a panel of various shape abnormalities such as elliptocytes, hemighosts, schizocytes and very rare stomatocytic cells. The missense mutation concerns a highly conserved residue among species, located in the region interacting with Calmodulin and responsible for the channel opening and the K⁺ efflux.

The functional experiments performed on Xenopus oocytes showed that the channel mutated on residue 352 is normally activated by Ca²⁺ influx, permits an efflux of K⁺ of increased intensity when compared to the wild-type (wt) channel and remains open and active during a prolonged period when compared to the normal channel. It is likely that the mutation, removing a positive charge in the Calmodulin binding domain of the Gardos channel, modifies interactions with this activating partner, resulting in a more active channel. Experiments on the human cell line HEK293 confirmed the higher current density for mutated KCNN4. Despite the mutation in the Calmodulin binding site, the trafficking properties of the mutant are similar and unaffected in cells with very different trafficking properties (HEK293 and Xenopus oocytes). These experiments showed that p.Arg352His mutation changes Ca²⁺ sensitivity of the channel that is activated by 10 times lower Ca²⁺ concentration. The anomaly in the kinetic of activation combined with a higher sensitivity to Ca²⁺ confers pathogenicity to p.Arg352His KCNN4.

The inventors have further shown that two other mutations in this Gardos channel, namely p.Val282Met (V282M) and p.Val282Glu (V282E) mutations, which participate in HX physiopathology (Andolfo et at, 2015; Glogowska et al., 2015), respectively lead to a gain in KCNN4 activity, as the R352H mutation.

The diagnostic of this disorders in these persons was prevented by the fact that two of the tests which could have led to biological diagnosis are no longer performed in routine laboratories: Osmotic Resistance has very often been replaced by the EMA test, which is normal in the present cases, and intra-erythrocytic K⁺ determination is currently no longer offered on a routine basis.

The provision of a new diagnostic test of hereditary xerocytosis is therefore of clinical interest.

In addition, there is currently no pharmacological treatment for this pathology.

The inventors have assessed the efficiency of Senicapoc, a derivative of the KCNN4 inhibitor clotrimazole, to inhibit mutated KCNN4. Senicapoc was tested in the past in a phase III study for the treatment of Sickle Cell disease and was proven, on this occasion, to be non-toxic (Ataga et al., 2011). Using transfected HEK cells and human red blood cells, the inventors have showed that Senicapoc is efficient in inhibiting KCNN4 current, thereby preventing K⁺ loss and dehydration in case of R352H mutation. Additionally the inventors have shown that a channel carrying the V282M mutation is as sensitive as the wild-type KCNN4 to Senicapoc, whereas the channel carrying the V282E mutation is much less sensitive. Thus, these results strongly support the Senicapoc to be considered as a therapy to treat red blood cell dehydration associated to at least R352H or V282M mutations in the Gardos channel.

Accordingly, the present invention provides an inhibitor of the Gardos channel (KCNN4 protein) for use in the treatment of hereditary xerocytosis.

The Gardos channel is a Ca²⁺ sensitive, intermediate conductance, potassium selective channel also referred to as KCa3.1 or KCNN4 (Maher and Kuchel, 2003). The Gardos channel can be a wild-type Gardos channel or a mutant Gardos channel, such as the Gardos channel (KCNN4 protein) variant p.Arg352His, p.Val282Met and p.Val282Glu, preferably p.Arg352His, p.Val282Met, more preferably p.Arg352His.

In a preferred embodiment, the Gardos channel is from human origin. The amino acid sequence of the wild-type human Gardos channel (KCNN4 protein) is available under accession number O15554 (GI:17366160) in the UniProtKB database, and referred herein to as SEQ ID NO: 2.

An inhibitor of the Gardos channel refers to a selective Gardos channel blocker that specifically inhibits the efflux of potassium from the erythrocytes.

An inhibitor of the Gardos channel can be identified by screening a collection of candidate compounds for their ability to specifically inhibit the efflux of potassium from the erythrocytes. Methods for measuring the inhibition of the efflux of potassium from the erythrocytes are known in themselves. Examples of such methods are described in Brugnara et al., 1993a and 1993b; Ellory et al., 1994. Both the percent inhibition of the Gardos channel and the IC₅₀ of an inhibitor of the Gardos channel can be assayed utilizing the methods described in Brugnara et al., 19931).

The potency of an inhibitor of the Gardos channel can be assayed using erythrocytes by a method such as that disclosed by Brugnara et al., 1993a.

Inhibitors of the Gardos channel include organic molecules, amino acids and antibodies.

The antibodies can be polyclonal or monoclonal antibodies. The term “antibody” or “antibodies” as used herein also encompasses functional fragments of antibodies, including fragments of chimeric, humanized, single chain antibodies or fragments thereof (e.g., Fv, Fab, Fab′ and F(ab′) 2 fragments). Suitable antibodies are those which are directed to KCNN4 protein (Gardos channel). Advantageously, said antibody is a monoclonal antibody, or fragment thereof.

In a preferred embodiment, the inhibitor of the Gardos channel is selected from the group consisting of imidazole antimycotics (Brugnara et al., 1996), such as clotrimazole (Brugnara et al., 1993a) metronidazole (Brugnara et al., 1993a), econazole (Brugnara et al., 1993a); arginine (Romero et al., 2002); Tram-34 (1-[(2-Chlorophenyl)diphenylmethyl]-1H-pyrazole) (Wulff et al., 2000); Charybdotoxin; Maurotoxin (Castle et al., 2002); nifedipine (Brugnara et al., 1993a); Nitrendipine (Brugnara et al., 1993a); inhibitors of calcium activated potassium flux that display selectivity and a potency towards the Gardos channel described in International Applications WO 00/50026, WO 2004/016221, WO 2005/113490 and WO 2006/084031, including senicapoc (ICA-17043; bis(4-fluorophenyl)phenyl acetamide; Ataga et al., 2008; 2009), 2,2-Bis(4-fluorophenyl)-N-methoxy-2-phenylacetamidine, 2-(2-Chlorophenyl)-2,2-diphenylacetaldehyde oxime, 2-(2-Chlorophenyl)-2,2-bis(4-fluorophenyl)-N-hydroxyacetamidine, 2,2,2-Tris(4-fluorophenyl)-N-hydroxyacetamidine, 2-(2-Fluorophenyl)-2-(4-fluorophenyl)-N-hydroxy-2-phenylacetamidine, phosphoric acid 3-(2-oxazolyl)-4-[3-(trifluoromethyl)phenylsulfonamido]phenyl monoester, N-[2-(4,5-Dihydrooxazol-2-yl)phenyl]-3-(trifluoromethyl)benzenesulfonamide, N-[4-Methoxy-2-(2-oxazolyl)phenyl]benzenesulfonamide, N-[4,5-Dimethoxy-2-(3-methyl-1,2,4-oxadiazol-5-yl)phenyl]-3-(trifluoromethyl)benzenesulfonamide, N-[2-(2-Furyl)phenyl]-3-(trifluoromethyl)benzenesulfonamide and N-[4-Methyl-2-(2-oxazolyl)phenyl]-3-(trifluoromethyl)benzenesulfonamide, preferably senicapoc (see also Stocker et al., 2003).

In a more preferred embodiment, the inhibitor of the Gardos channel is senicapoc.

The inhibitor of the Gardos channel can be administered by itself, or mixed with suitable carriers or excipient(s). It can be used systemically. One can use any formulation suitable for systemic administration.

As used herein, the terms “treatment” or “treating” includes the administration of an inhibitor of the Gardos channel as defined above to a subject who has hereditary xerocytosis, with the purpose to alleviate, relieve, alter, remedy, ameliorate, improve or affect this disorder.

The subject is preferably a human subject, more preferably a human subject who is a carrier for a missense mutation selected from the group consisting of c.1055G>A, c.844G>A and c.845T>A, in the KCNN4 gene encoding the Gardos channel, resulting respectively in an amino acid change from arginine to histidine in codon 352 (p.Arg352His), in an amino acid change from valine to methionine in codon 282 (p.Val282Met) or in an amino acid change from valine to glutamine in codon 282 (p.Val282Glu), preferably c.1055G>A or c.844G>A, more preferably the missense mutation c.1055G>A.

The nucleic acid sequence of the wild-type human KCNN4 gene encoding the Gardos channel (Map:19q13.2) is available under the accession number NC_000019.10 (G1:568815579) in the NCBI GenBank database.

The nucleic acid sequence of the mRNA (cDNA) encoded by the wild-type human KCNN4 gene is available under the accession number NM_002250.2 (GI:25777651) in the NCBI GenBank database, referred herein to as SEQ ID NO: 1.

The amino acid sequence of the wild-type human Gardos channel (KCNN4 protein) is available under accession number 015554 (GI:17366160) in the UniProtKB database or NM 002250.2 (GI:25777651) in the NCBI GenBank database, referred herein to as SEQ ID NO: 2.

Methods for identifying said mutation are described below.

In a particular embodiment the present invention provides senicapoc (ICA-17043; bis(4-fluorophenyl)phenyl acetamide) for use in the treatment of hereditary xerocytosis in a human subject who is a carrier for a missense mutation selected from the group consisting of c.1055G>A, c.844G>A and c.845T>A, in the KCNN4 gene encoding the Gardos channel, resulting respectively in an amino acid change from arginine to histidine in codon 352 (p.Arg352His), in an amino acid change from valine to methionine in codon 282 (p.Val282Met) or in an amino acid change from valine to glutamine in codon 282 (p.Val282Glu), preferably selected from c.1055G>A and c.844G>A, more preferably the missense mutation c.1055G>A.

The present invention also provides a method for treating hereditary xerocytosis, comprising administering to a subject in need thereof an effective amount of an inhibitor of the Gardos channel (KCNN4 protein) as defined above.

The present invention also provides the use of an inhibitor of the Gardos channel (KCNN4 protein) as defined above for the preparation of a medicament for treating hereditary xerocytosis.

The present invention also provides a method for genotyping, in vitro, the KCNN4 gene in a human subject comprising the steps of:

(a) isolating mRNA or genomic DNA from a nucleic acid sample obtained from said subject,

(b) determining the nucleotide present at position c.1055, c.844 or c845, preferably c.1055, of the KCNN4 gene encoding the Gardos channel.

As used herein, the term “determining the nucleotide corresponding to the nucleotide present at position c.1055, c.844 or c845 of the KCNN4 gene encoding the Gardos channel” refers to determining the nucleotide corresponding to the nucleotide present at position c.1055, c.844 or c845 of the KCNN4 gene encoding the Gardos channel, either in said isolated mRNA or genomic DNA.

Said subject is suffering or not from hereditary xerocytosis.

Methods for obtaining a nucleic acid sample from a subject are well known in the art. Methods for isolating mRNA or genomic DNA from a subject are also well known in the art.

Advantageously, the mRNA or genomic DNA can be obtained from a blood sample from said subject, in particular from reticulocytes from said subject for isolating mRNA or from white blood cells from said subject for isolating genomic DNA.

Methods for determining said nucleotide in step (b) comprise the methods for detecting a single nucleotide polymorphism (SNP) which are well known in this art.

Methods for detecting SNPs have been described in the prior art, including selective hybridization techniques (e.g., reverse dot blot, Southern blot for DNAs, Northern blot for RNAs,), selective amplification, nucleic acid sequencing, restriction fragment length polymorphism (RFLP), amplified fragment length polymorphism (AFLP), ligation chain reaction (LCR), mass spectrometry (see for review Kaplan and Delpech, 2007).

In particular, to determine a SNP in mRNA or genomic DNA, it may be necessary to amplify the corresponding mRNA or genomic region respectively. For this, it can be used PCR primers whose nucleotide sequences may be obtained from the sequences containing the SNP to be amplified. The PCR amplified fragments can then be analyzed by sequencing (e.g., Sanger sequencing) or hybridization techniques. The PCR amplified fragments can also be analyzed on mass spectrometer through specific extension primers distinguishing the two variants (wild-type or variant) known at the polymorphic site.

By way of examples, a set of PCR primers as defined above include the set of PCR primers of SEQ ID NO: 5 and SEQ ID NO: 6.

A SNP in mRNA or genomic DNA can also be determined by reverse dot blot. One can use the probe of SEQ ID NO: 3 to determine the wild-type sequence and the probe of SEQ ID NO: 4 to determine the c.1055G>A variant (mutant) sequence.

Methods for genotyping the mutations c.844 or c845 of the KCNN4 gene encoding the Gardos channel are described in Andolfo et al., 2015 and Glogowska et al., 2015.

According to the method for genotyping it can be deduced that the subject is suffering from hereditary xerocytosis if the nucleotide present at position c.1055 of the KCNN4 gene encoding the Gardos channel is adenine (A), or if the nucleotide present at position c.844 of the KCNN4 gene encoding the Gardos channel is adenine (A), or if the nucleotide present at position c.845 of the KCNN4 gene encoding the Gardos channel is adenine (A).

The present invention also provides an in vitro method of diagnosing the presence of or predisposition to hereditary xerocytosis in a human subject, comprising the step of:

(i) providing a biological sample from said subject and

(ii) detecting in said biological sample the presence of a missense mutation selected from the group consisting of c.1055G>A, c.844G>A and c.845T>A, preferably selected from c.1055G>A and c.844G>A, more preferably the missense mutation c.1055G>A, in the KCNN4 gene encoding the Gardos channel, or a missense mutation selected from the group consisting of p.Arg352His, p.Val282Met and p.Val282Glu, preferably selected from p.Arg352His and p.Val282Met, more preferably the missense mutation p.Arg352His, in the Gardos channel (KCNN4 protein),

the presence of said mutation constituting a marker of a hereditary xerocytosis or a predisposition to hereditary xerocytosis in said subject.

In a preferred embodiment of step (ii), the presence of a missense mutation selected from c.1055G>A, c.844G>A and c.845T>A, preferably selected from c.1055G>A and c.844G>A, more preferably the missense mutation c.1055G>A in the KCNN4 gene encoding the Gardos channel is detected.

As used herein the term “detecting the presence of the missense mutation c.1055G>A, c.844G>A or c.845T>A in the KCNN4 gene” refers to detecting the presence of the mutation corresponding to missense mutation c.1055G>A, c.844G>A or c.845T>A in the KCNN4 gene, either in mRNA or genomic DNA from a nucleic acid sample obtained from said subject.

The presence of the missense mutation c.1055G>A, c.844G>A or c.845T>A in the KCNN4 gene encoding the Gardos channel can be detected by genotyping the KCNN4 gene in said human subject as described above.

Methods for determining a point mutation in a protein are well known in this art (see for review Kaplan and Delpech, 2007). The presence of the p.Arg352His, p.Val282Met or p.Val282Glu mutation in the Gardos channel (KCNN4 protein) can be detected by protein sequencing or binding to a ligand (such as an antibody) specifically directed to the Gardos channel (KCNN4 protein) variant p.Arg352His, p.Val282Met or p.Val282Glu respectively, in particular by western blot using antibodies specifically directed to the Gardos channel (KCNN4 protein) variant p.Arg352His, p.Val282Met or p.Val282Glu respectively, preferably by protein sequencing.

The present invention also provides a method of diagnosing and treating hereditary xerocytosis in a subject, comprising the steps of:

(i) providing a biological sample from said subject,

(ii) detecting in said biological sample whether a missense mutation selected from the group consisting of c.1055G>A, c.844G>A and c.845T>A, preferably selected from c.1055G>A and c.844G>A, more preferably the missense mutation c.1055G>A, in the KCNN4 gene encoding the Gardos channel is present, or a missense mutation selected from the group consisting of p.Arg352His, p.Val282Met and p.Val282G1u, preferably selected from p.Arg352His and p.Val282Met, more preferably the missense mutation p.Arg352His, in the Gardos channel (KCNN4 protein) in present, as defined above,

(iii) diagnosing the subject with a hereditary xerocytosis when the presence of a mutation as defined in step (ii) in the biological sample is detected, and

(iv) administering an effective amount of an inhibitor of the Gardos channel (KCNN4 protein), preferably senicapoc, as defined above to the diagnosed subject.

The present invention also provides a kit for diagnosing a hereditary xerocytosis comprising the probe of SEQ ID NO: 3 and/or the probe of SEQ ID NO: 4.

The present invention also provides the use of the probe of SEQ ID NO: 3 and/or the probe of SEQ ID NO: 4 for in vitro diagnosing a hereditary xerocytosis in a human subject.

The present invention also relates to methods for screening inhibitors of the Gardos channel. Such inhibitors are useful as selective Gardos channel blocker that specifically inhibits the efflux of potassium from the erythrocytes, and therefore for treating hereditary xerocytosis.

The methods include binding assays and/or functional assays, and may be performed in vitro, in cell systems (yeast, bacteria, Xenopus oocyte) or in animals, involving the human Gardos channel (KCNN4 protein) variant p.Arg352His, p.Val282Met or p.Val282Glu, preferably p.Arg352His or p.Val282Met, more preferably p.Arg352His.

For cell systems, cells can be native, i.e., cells that normally express the Gardos channel (KCNN4 protein) variant p.Arg352His p.Val282Met or p.Val282Glu polypeptide, as a biopsy or expanded in cell culture. Preferably, these native cells are derived from erythrocytes. Alternatively, cells are recombinant host cells, in particular Xenopus laevis oocytes, expressing the Gardos channel (KCNN4 protein) variant p.Arg352His, p.Val282Met or p.Val282G1u.

The present invention therefore provides an in vitro method for screening a biologically active inhibitor of a human Gardos channel (KCNN4 protein) variant selected from the group consisting of p.Arg352His, p.Val282Met and p.Val282Glu, preferably the variant p.Arg352His or p.Val282Met, more preferably the variant p.Arg352His, said method comprising contacting in vitro a test compound with the human Gardos channel (KCNN4 protein) variant p.Arg352His, p.Val282Met or p.Val282G1u, and determining the ability of said test compound to prevent ion conductance through the channel when compared to the wild-type human Gardos channel (KCNN4 protein) of SEQ ID NO: 2, wherein preventing ion conductance through the channel when compared to the wild-type human Gardos channel (KCNN4 protein) of SEQ ID NO: 2 provides an indication as to the ability of the compound to inhibit the human Gardos channel (KCNN4 protein) variant p.Arg352His, p.Val282Met or p.Val282Glu.

In a preferred embodiment of said method, the method comprises expressing a plasmid containing the mutated cDNA encoding the human Gardos channel (KCNN4 protein) variant p.Arg352His, p.Val282Met or p.Val282Glu in Xenopus laevis oocytes and measuring the current voltage in the presence of the test compound; a decrease in the conductance indicating that said test compound inhibits the human Gardos channel (KCNN4 protein) variant p.Arg352His, p.Val282Met or p.Val282Glu.

In another preferred embodiment of said method, erythrocytes expressing the Gardos channel (KCNN4 protein) variant p.Arg352His, p.Val282Met or p.Val282Glu polypeptide are exposed to a test compound and a Rb-containing medium. The initial rate of ⁸⁶Rb transport can be calculated from a parameter such as the linear least square slope of ⁸⁶Rb uptake by the erythrocytes. Inhibitory constants can be calculated by standard methods using computer-assisted nonlinear curve fitting.

A method for measuring current voltage in Xenopus laevis oocytes is described in Example I below.

In addition to the above features, the invention further comprises other features which will emerge from the following description, which refers to the identification of the p.Arg352His mutation in the Gardos channel and its association with chronic hemolysis and dehydrated cells, and the use of senicapoc for the treatment of hereditary xerocytosis caused by mutations in the Gardos channel, as well as to the appended figures:

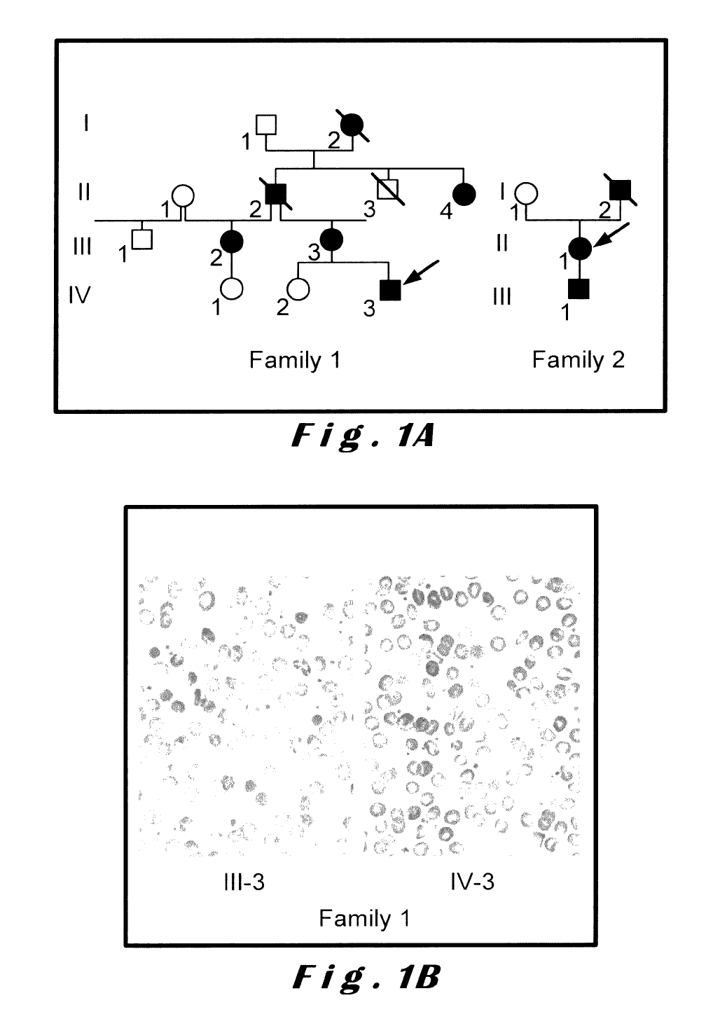

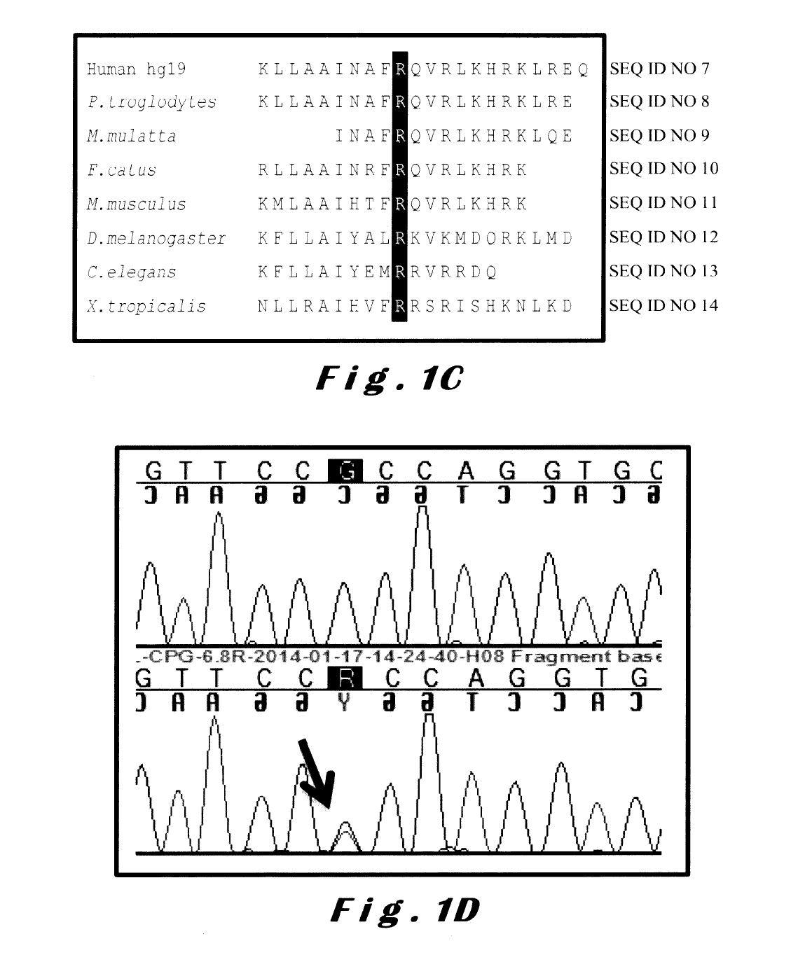

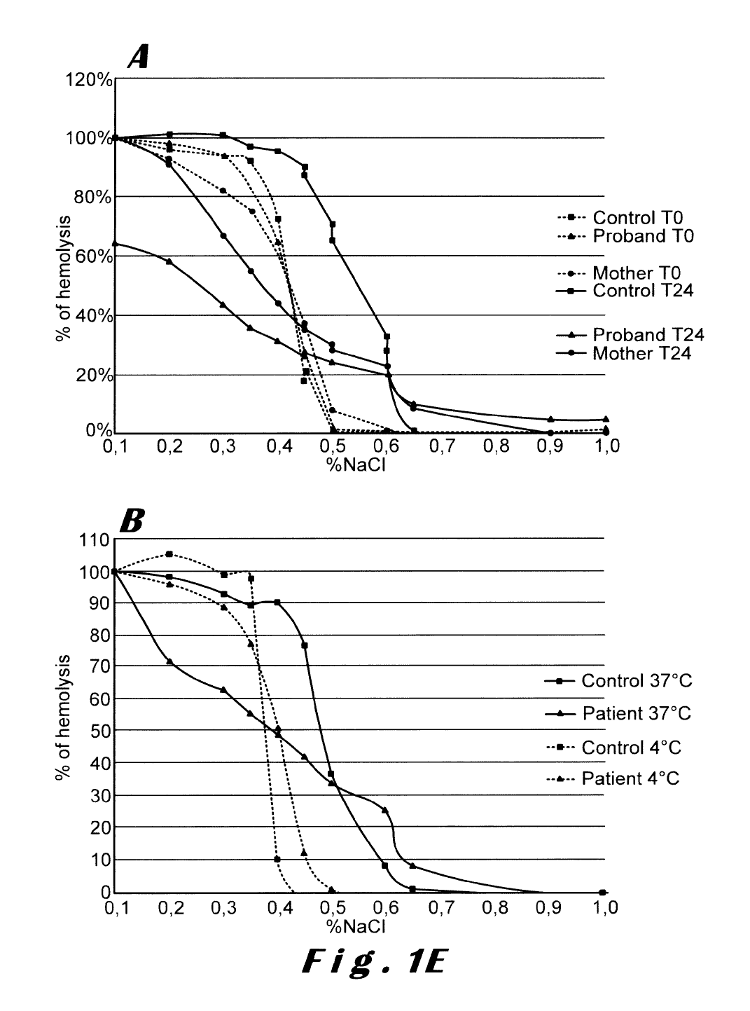

FIG. 1: Red blood cell and DNA investigations. A: Family pedigrees showing mutation segregation; B: Blood film smears (MGG) for the proband 1 and his mother; C: Multiple interspecies protein sequence alignment of KCNN4 in the region of residue 352; D: KCNN4 transcript sequencing: upper panel: wild type sequence; bottom panel: transcript with mutation c.1055G>A (p.Arg352His). E: Red cell Osmotic fragility test using osmolar gradient ranging from 0.1 to 1% of NaCl solution. A: at TO and at 37° C. for a Control, Mother and Proband from Family 1. B: after T24h incubation at 4° or 37° C. for a control and the proband.

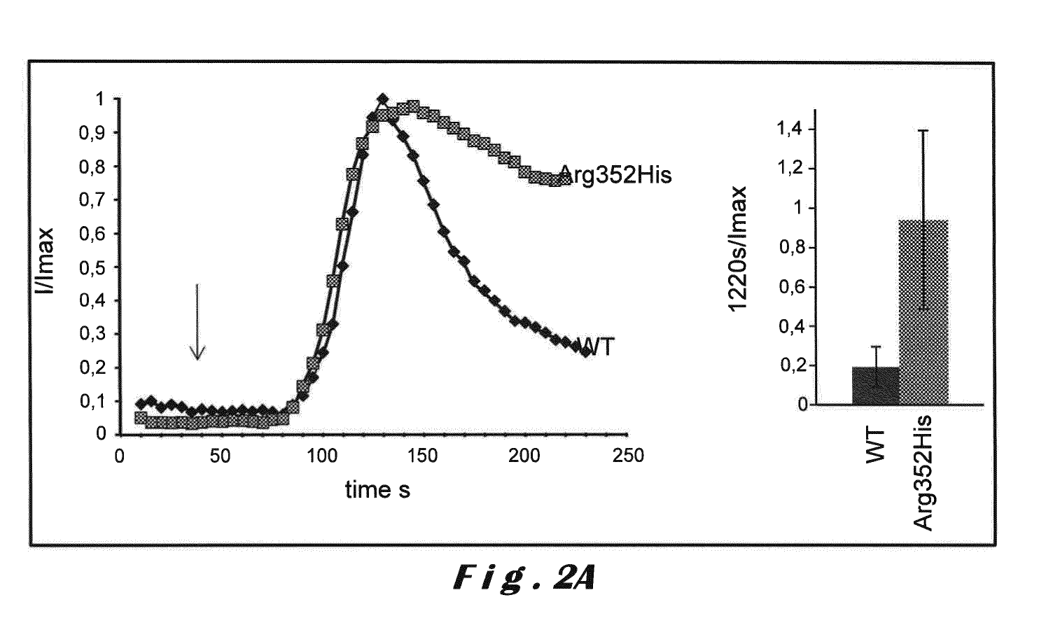

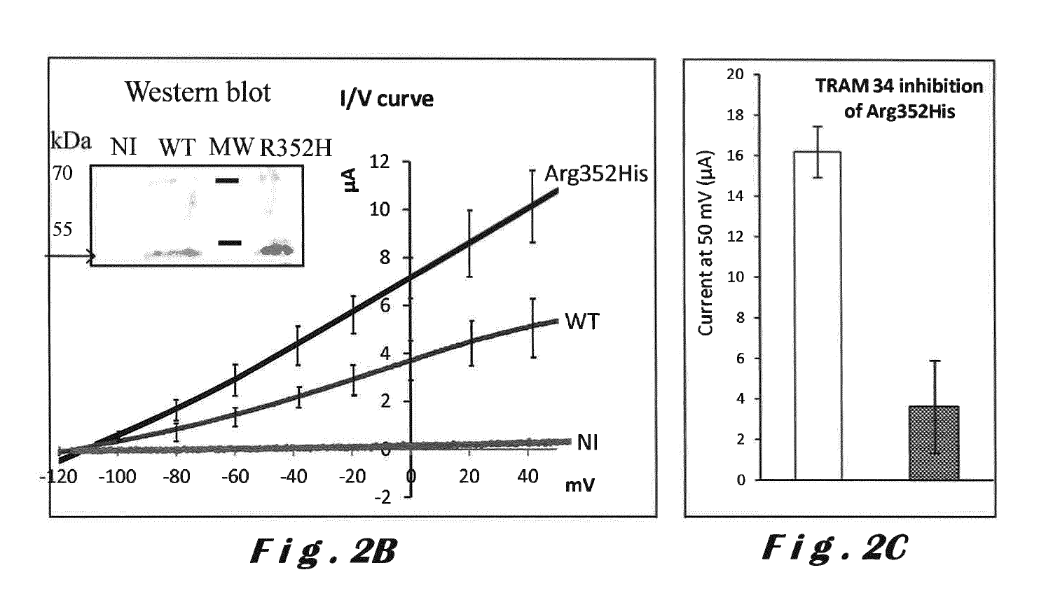

FIG. 2: Functional analysis of the Gardos channel variant p.Arg352His. A: Activation kinetic. For oocytes expressing WT KCNN4 or p.Arg352His KCNN4, the current at 0 mV was plotted as a function of time (left panel). The maximal intensity of the current being different between WT and mutated KCNN4, a ratio between I at different times and the Imax was calculated for each condition and plotted as a function of time. Data are means of 15 (WT) or 22 (p.Arg352His) oocytes coming from 3 different batches. The arrow indicates the opening of calcium ionophore perfusion. The bar graph (right panel) quantifies the remaining current at 220 s in oocytes expressing WT or p.Arg352His KCNN4. The current at 220 s was divided by Imax (at about 135 s) for each recording. Data are means+/−sem of 15 (WT) or 22 (p.Arg352His) oocytes. Statistical analysis were done using the Mann and Whitney test, the two bars are different with a risk of 0.2% (bidirectional). B: Current-voltage curves of WT and mutated KCNN4 in oocyte membranes with quantification. I/V curves correspond to the maximal current recorded for WT or p.Arg352His KCNN4 expressing oocytes (around 135 s) in gluconate medium with 1 μM A23187. Data are means of ramps recorded on 15 (WT) or 22 (p.Arg352His) oocytes. NI are control (non-injected) oocytes (n=4). Inset: western blot detection of WT and mutated KCNN4 indicated by the arrow (around 50 kDa). C: TRAM-34 inhibition: once the maximal current was reached in oocytes expressing p.Arg352His mutant, 10 μM TRAM-34 was added. This induced a rapid current decrease. The mean value of maximal currents at 50 mV was calculated (white bar) and compared to the mean value of minimal currents at 50 mV after TRAM-34 addition (grey bar). Data are means of 4 oocytes +/−sem.

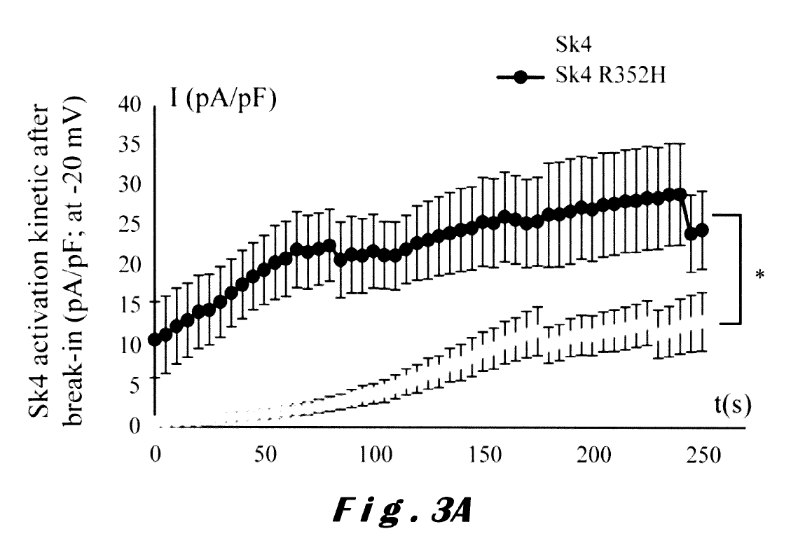

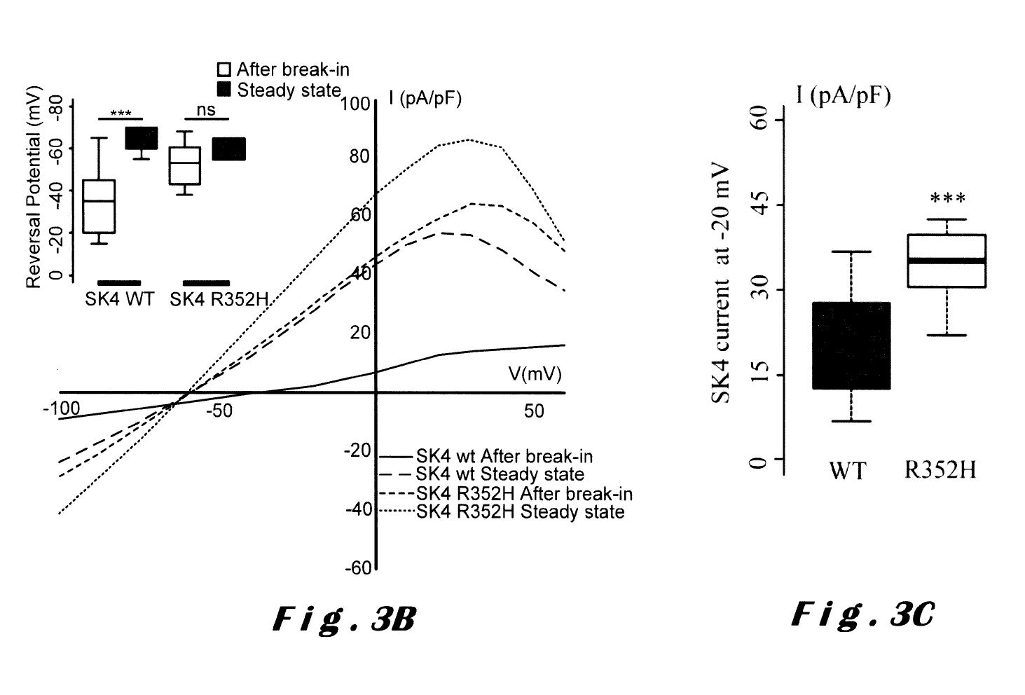

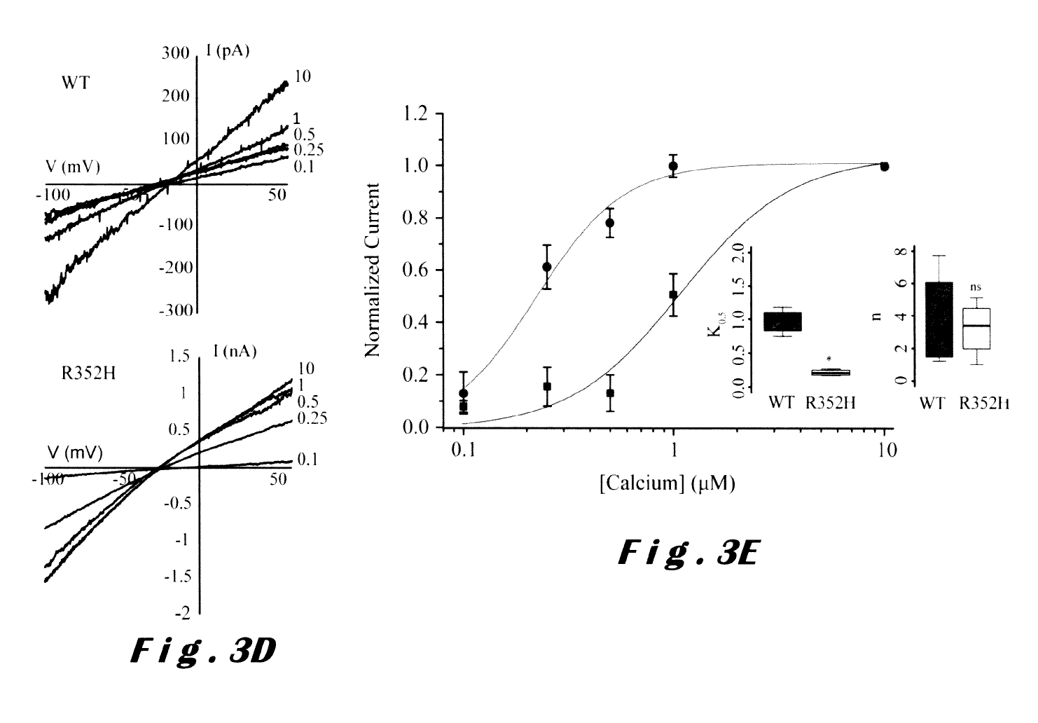

FIG. 3: KCNN4 expression in HEK293 cells. A: Activation kinetic of wt and p.Arg352His KCNN4 recorded in whole cell configuration. WT KCNN4 or p.Arg352His KCNN4 were expressed in HEK293 cells and then subjected to patch-clamp experiment in whole cell configuration. Current were recorded immediately after break-in using a 150 ms voltage ramp protocol from −120 to +80 mV from an holding potential of −60 mV. The current at −20 mV was plotted as a function of time. Values are mean +SEM of 12-8 experiments. B: Representative current/voltage curves for HEK293 expressing wt or p.Arg352His KCNN4. Inset (upper left) represents reversal potentials just after break-in and at the steady state. Values are represented as a Tukey's plot (n=12-8) Statistical analyses were done using Kruskal and Wallis test followed by a Tukey post-hoc test. C: Tukey's plots showing current density at −20 mV in wt and mutated sk4 (n=12-8; ***p<0.001). Statistical analysis was performed using a Mann and Whitney test. Bs: Representative traces of Ca²⁺-dependent activation of wt and p.Arg352His KCNN4 current recorded in an inside-out macropatch configuration. Currents were elicited by 150 ms voltage ramps from −120 to +80 mV. Each trace corresponds to a different concentration of Ca²⁺ indicated on the right hand side of the I/V. E: Normalized K⁺ current measured at −45 mV in response to [Ca²⁻]i was plotted as a function of [Ca²⁺]I for wt (squares) and p.Arg352His mutated KCNN4 (circles). The experimental values (mean ±SEM) were fitted with the Hill equation (Origin software (Northampton, Mass.)):

$$Y = \frac{Y {max [ {Ca}^{2 +} ]}_{i}^{n}}{{( {K}_{0.5} )}^{n} + {[ {Ca}^{2 +} ]}_{i}^{n}}$$

where Y is relative KCNN4 current at −45 mV (I/Imax) for each [Ca²⁺], Ymax is the maximum current (Imax), K0.5 is the apparent dissociation constant, and n is the Hill coefficient. Insert (lower right) show Hill equation parameter K0.5 and nh. Values are represented as Tukey's plot (n=4, *p<0.05). Statistical analysis was performed using a Mann and Whitney test.

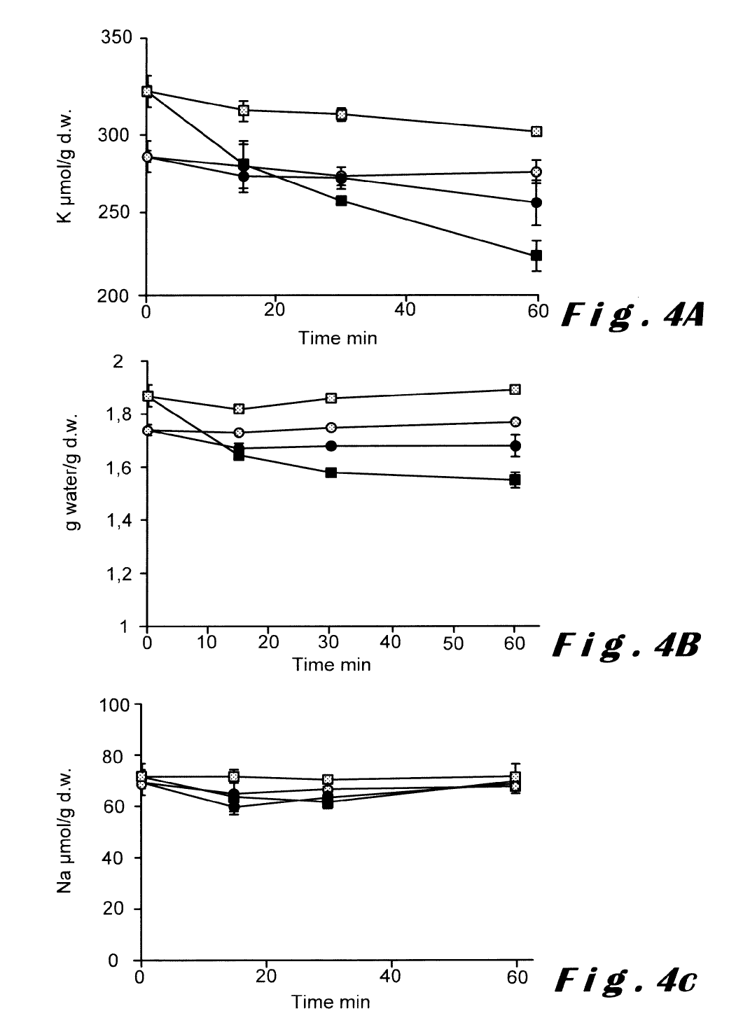

FIG. 4: Cation contents and cell volume as a function of incubation time with vanadate. A: K⁺ content, B: cell water and C: Na⁺ contents in red cells incubated with 5 mM vanadate (black circles for control red cells, black squares for patient red cells) or 5 mM vanadate with 10 μM TRAM-34 (grey circles for control red cells and grey squares for patient red cells). Data in μmol per g of dry weight, are mean ±S.D., n=3.

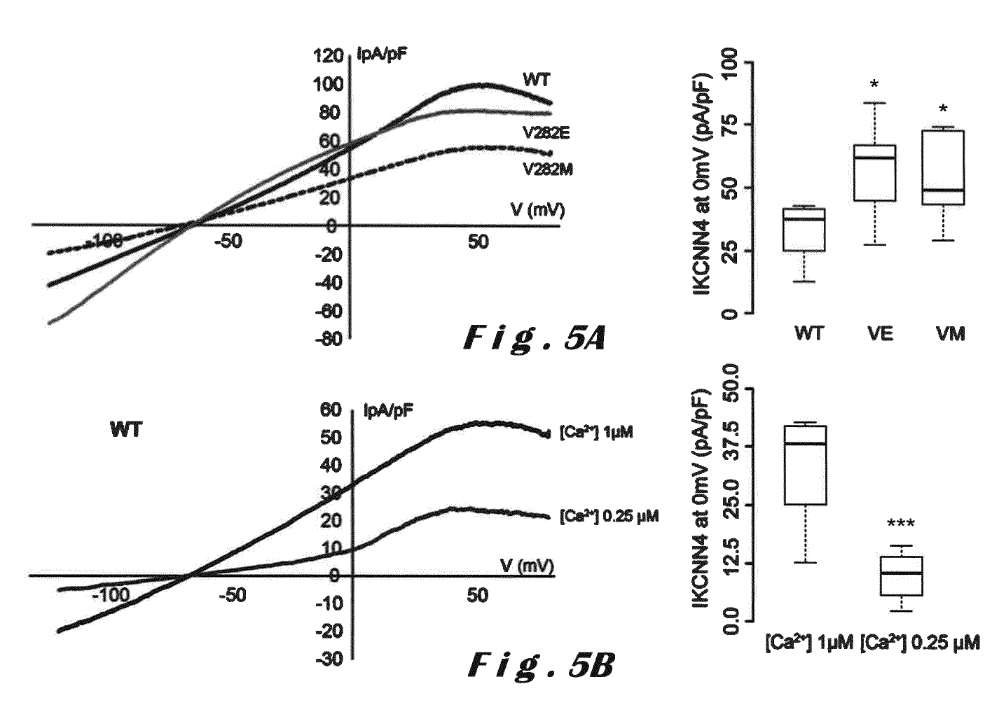

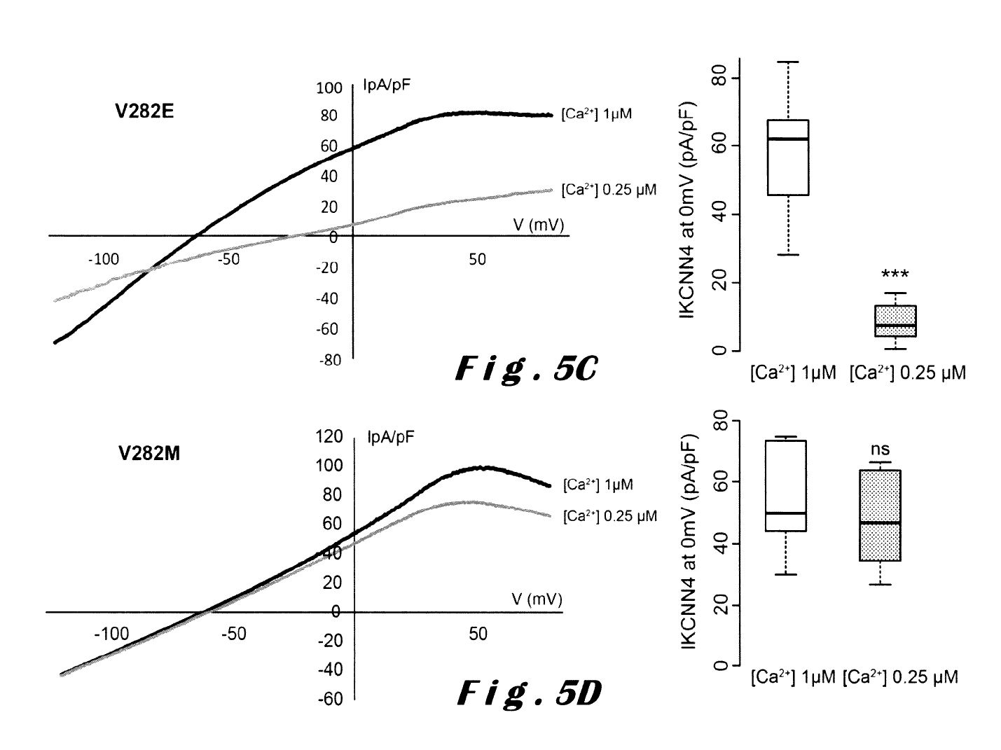

FIG. 5: Characterization of KCNN4 mutants in HEK293 cells. A: WT KCNN4, V282E KCNN4, or V282M KCNN4, were expressed in HEK293 cells and then subjected to patch clamp experiment in whole cell configuration using a 150 ms voltage ramp protocol from −120 to +80 mV from an holding potential of −60 mV. Mean current/voltage curves from 8 to 10 experiments are represented on the left hand side. On the right hand panel, values are represented as Tukey's box plots showing current density measured at 0 mV in cell transfected with WT and mutated KCNN4 (V282E and V282M). Statistical analyses were done using Kruskal and Wallis test followed by a Tukey post-hoc test. (n=8-10; *p<0.05). B: WT KCNN4 were expressed in HEK293 cells and then subjected to patch clamp experiment as described in A. Mean current/voltage curves from cells recorded with an intracellular solution containing 1 μM free Ca²⁺ (n=8), or containing 0.25 μM free Ca²⁺ (n=4) are represented on the left hand side. On the right panel, values are represented as a Tukey's plot. Statistical analyses were done using Mann and Withney test (n=4-8, ***p<0.001). C: V282E KCNN4 were expressed in HEK293 cells and then subjected to patch clamp experiment as described in A. Mean current/voltage curves from cells recorded with an intracellular solution containing 1 μM free Ca²⁺ (n=10), or containing 0.25 μM free Ca²⁺ (n=8) are represented on the left hand side. On the right panel, values are represented as a Tukey's plot. Statistical analyses were done using Mann and Withney test (n=8-10, ns: non-significant). D: V282M KCNN4 were expressed in HEK293 cells and then subjected to patch clamp experiment as described in A. Mean current/voltage curves from cells recorded with an intracellular solution containing 1 μM free Ca²⁺ (n=8), or containing 0.25 μM free Ca²⁺ (n=8) are represented on the left hand side. On the right panel, values are represented as a Tukey's plot. Statistical analyses were done using Mann and Whitney test (n=8, p<0.001).

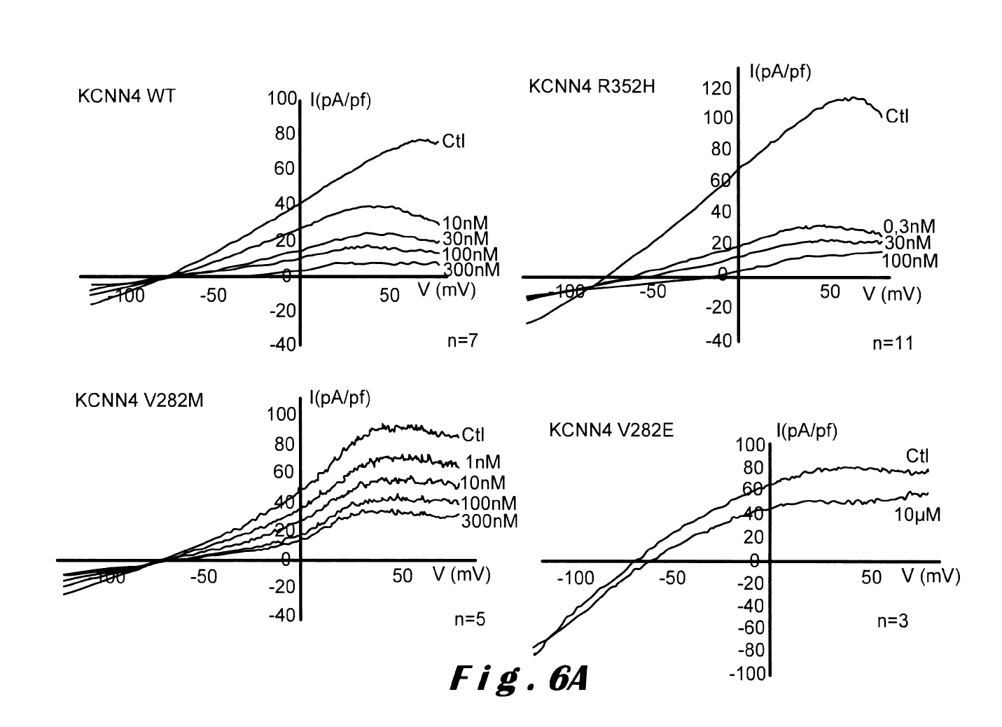

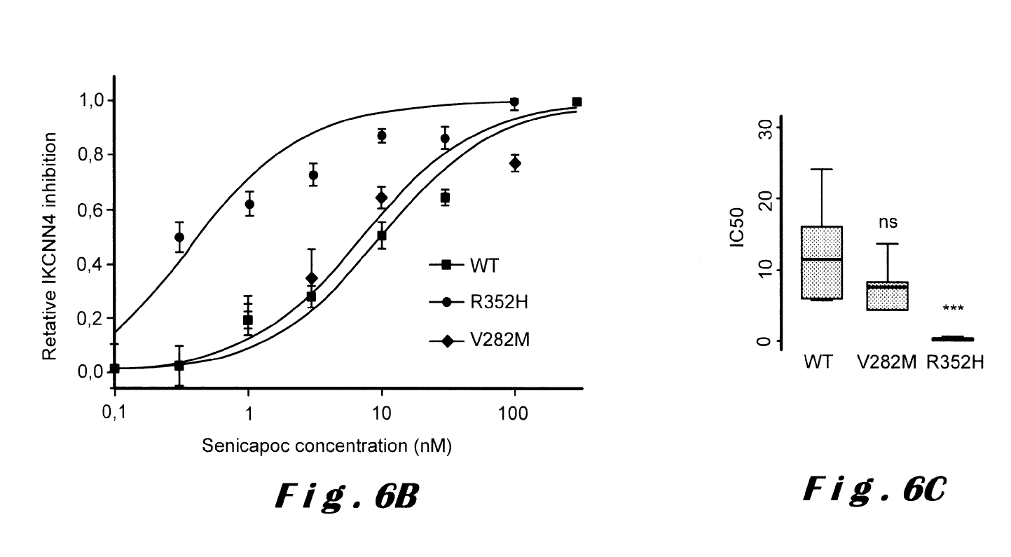

FIG. 6: Effect of Senicapoc on KCNN4 mutants in HEK293 cells. A: Representative traces showing dose-dependent inhibition of WT and mutated KCNN4 (R352H, V282M and V282E) by Senicapoe (n=number of cells recorded in each condition). Currents recorded in a whole cell patch clamp configuration were elicited by 150 ms voltage ramps from −120 to +80 mV from a holding potential of −60 mV. Each trace corresponds to a different concentration of Senicapoc indicated on the right hand side of the I/V. B: Normalized K⁺ currents measured at 0 mV in response to [Senicapoc] was plotted as a function of [Senicapoc] for WT KCNN4 (grey squares), R352H mutated KCNN4 (black squares) and V282M mutated KCNN4 (grey circles). The experimental values (mean ±SEM) were fitted using the Hill equation. C: Tukey's box plots showing IC50 values for each condition (n=5-11, *p<0.05). Statistical analysis was performed using a Kruskal and Wallis test followed by a Tukey post-hoc test.

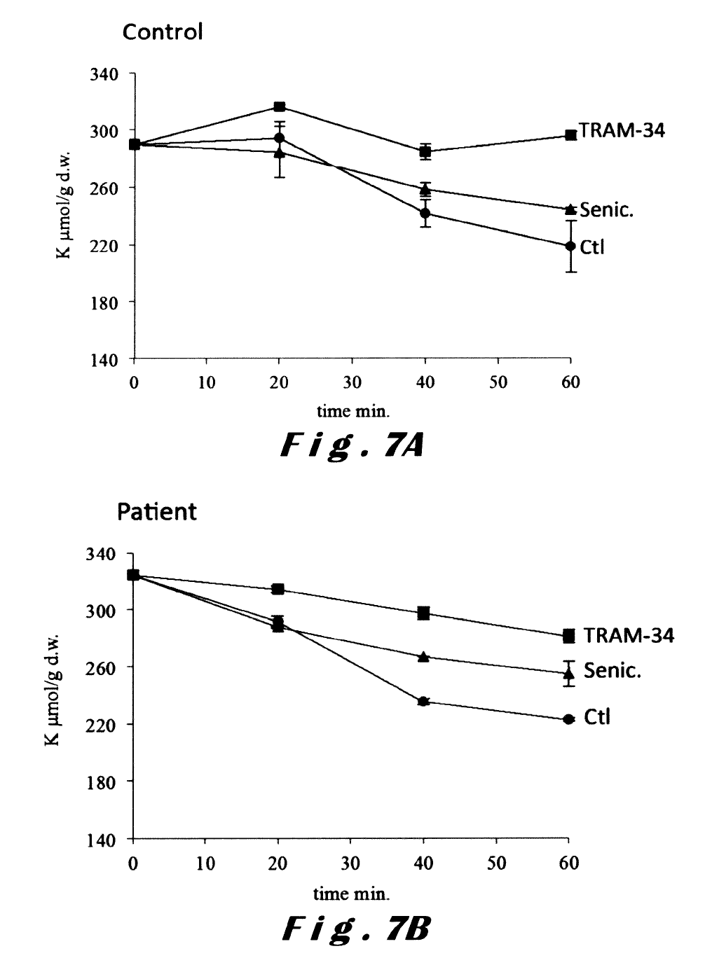

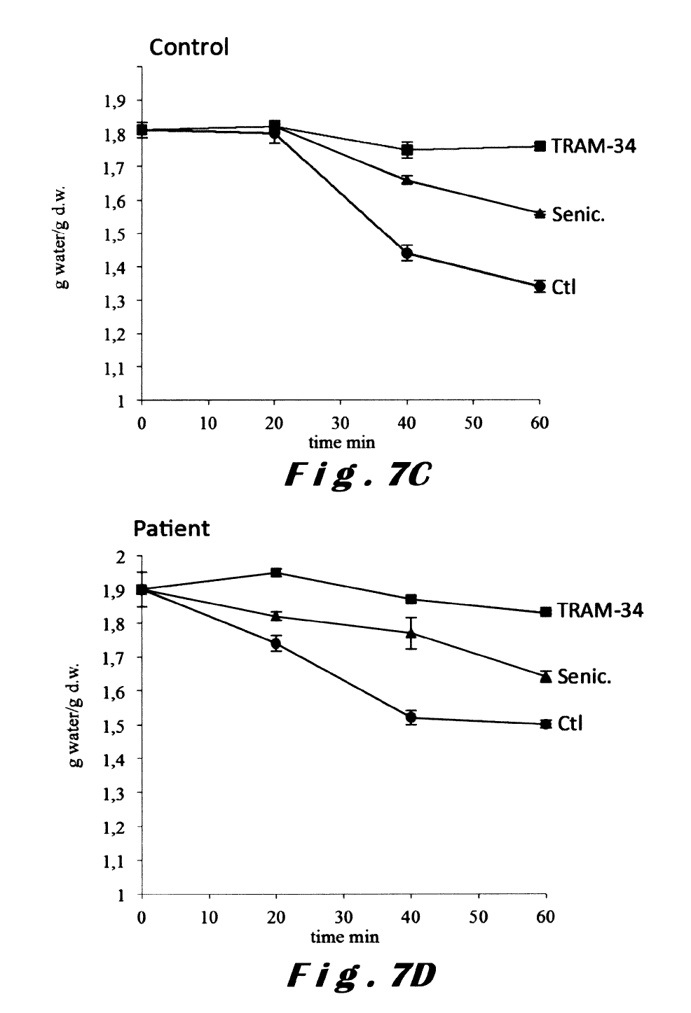

FIG. 7: Effect of Senicapoc and TRAM-34 on red blood cells with KCNN4 R352H mutation. Kinetic of net K⁺ fluxes in control (A) or patient (B) red blood cells and water contents in control (C) or patient (D) red blood cells. 5 μM vanadate was added at t=0 on red cells in absence (circles) or presence of TRAM-34 10 μM (squares) or Senicapoc 0.4 μM (triangles). Data are means±s.e.m. of three samples coming from one representative experiment over 3 with blood from a single patient and two different controls.

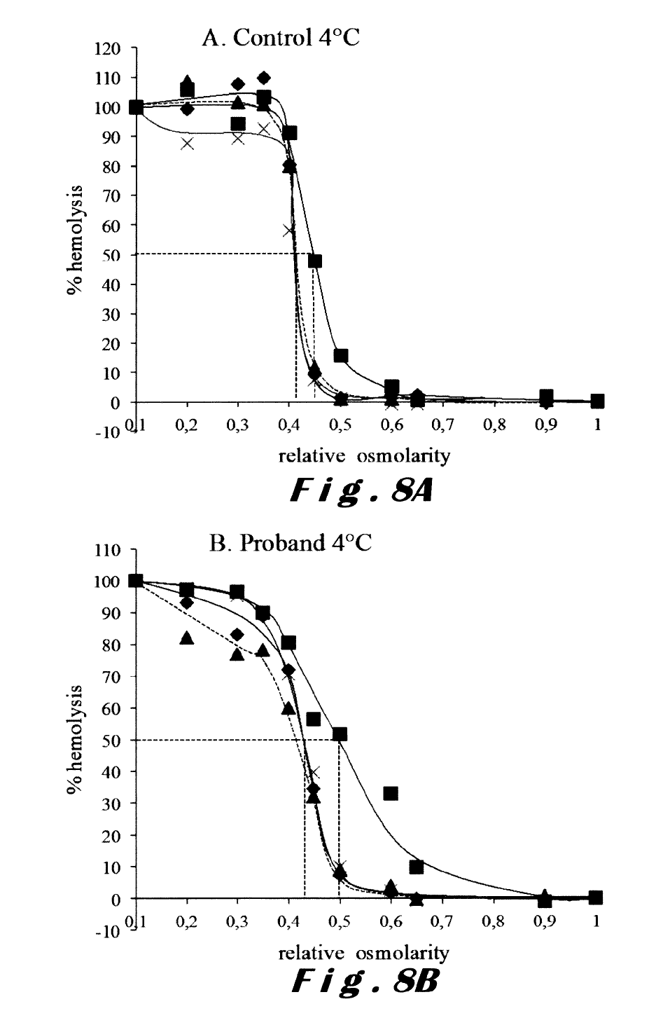

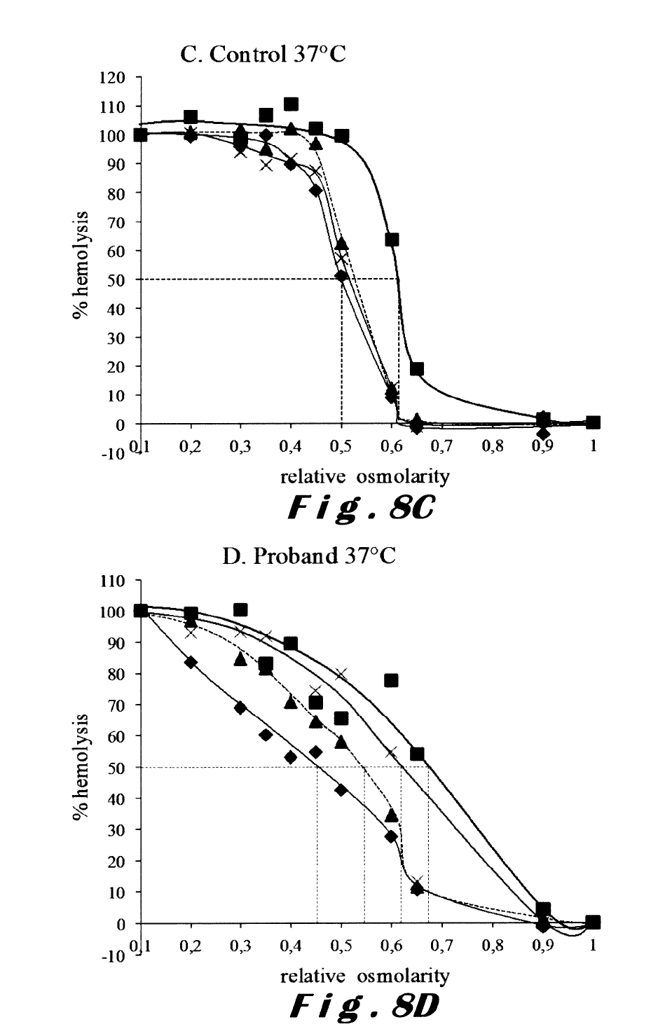

FIG. 8: Osmotic resistance of control or patient red blood cells with KCNN4 R352H mutation. Blood was incubated for at 4° C. (A control-B patient) or 37° C. (C control-D patient). The incubation was done in absence (diamonds) or presence of TRAM-34 10 μM (grey squares), Senicapoc 0.4 μM (grey triangles, dashed lines) or 4 μM (light grey crosses). Data are representative of 2 to 3 different experiments with blood from a single patient and two different controls.

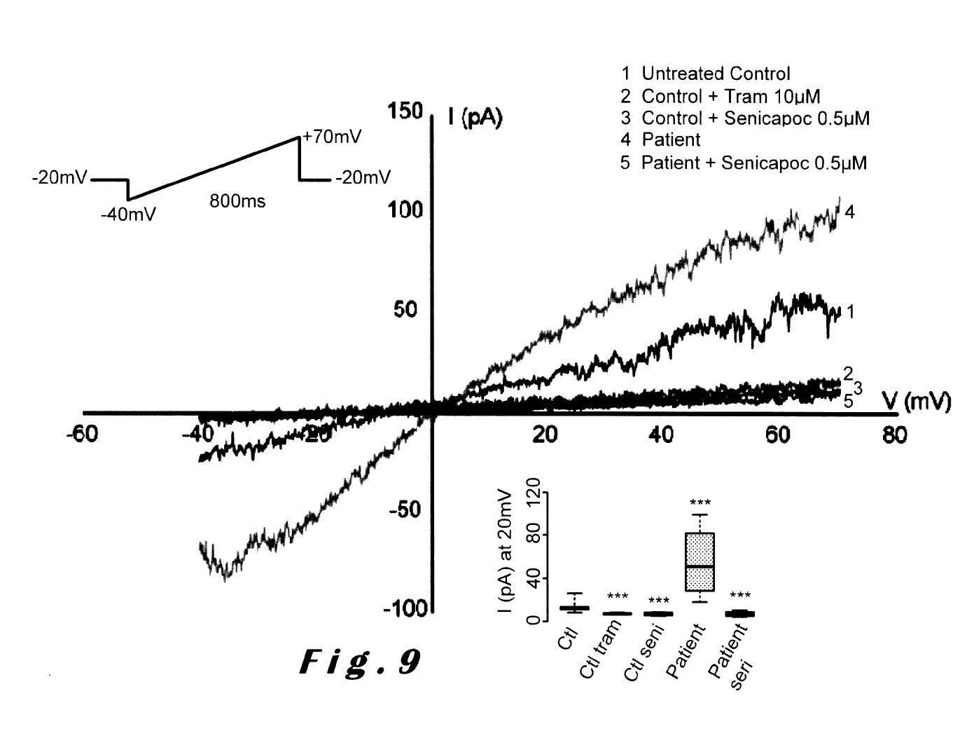

FIG. 9: Effect of Senicapoc on KCNN4 current in human red blood cells. Endogenous KCNN4 current was recorded using the whole cell patch clamp configuration. Currents were elicited by a 800 ms voltage ramp protocol from −40 to +70 mV from an holding potential of −20 mV. Red blood cells were pre-incubated with TRAM-34 (10 μM) or Senicapoc (0.5 μM) for 5 min and then submitted to patch clamp experiments. Representative current/voltage curves from patient (in yellow n=4), treated with Senicapoc (in green n=6) or controls (in grey n=5), treated with TRAM-34 (in orange n=4) or with Senicapoc (in blue n=3), are shown, Inset (lower right) shows quantification of currents recorded from red blood cells. Values are represented as a Tuckey's plot (***p<0.001).

### EXAMPLE I

### Identification of the p.Arg352His Mutation in the Gardos Channel Associated with Hereditary Xerocytosis

I. Material and Methods

Hematological tests: An osmotic fragility test, based on the observation of the fragility of red blood cells in hypotonic saline solutions, was performed immediately after sampling and after 24 hours' incubation at 4° or 37° C.

NMR: NMR experiments were performed on a 400 AVANCE wide-bore spectrometer (Bruker Biospin, Billerica, Mass.), using stimulation by the ionophore A23187 (Sigma Aldrich).

NGS Sequencing: Exome sequencing was performed after exome enrichment using Ion AmpliSeq™ (Thermo Fisher Scientific Inc., Waltham, Mass. USA), template preparation using the Ion PI™ Template OT2 200 Kit v2 on the Ion OneTouch™ 2 System and sequencing using the Ion PI™ Chip Kit v2 and Ion PI™ Sequencing 200 Kit v2 on the Ion Proton™ Sequencer (Thermo Fisher Scientific Inc., Waltham, Mass. USA). Raw data were first aligned with the provided software suite to generate BAM files. The coverage and sequencing depth analysis were computed using the BEDtools suite v2.17 (Quinlan and Hall, 2010) and in-house scripts. Variants were identified using the Torrent Browser Variant caller (version 4.0.2), annotated and prioritized with the in-house “VarAFT” system that includes Annovar (Wang et al., 2010).

The mutation was confirmed on DNA samples and KCNN4 transcripts from fresh reticulocytes by Sanger sequencing (3500XL Genetic AnalyzerR, Life Technologies, Carlsbad, Calif.).

Expression in Xenopus oocytes: Plasmid pcDNA3KCNN4-HA (Joiner et al,, 2001) was used to introduce the point mutation p.Arg352His by PCR. A Hemagglutinin tag (HA) was present in the C-terminal end of KCNN4 (Joiner et al., 1997). Female Xenopus laevis were anaesthetized with MS222 according to the procedure recommended by ethics committee of the applicants. Oocytes were harvested and injected as previously published (Barneaud-Rocca et al., 2011).

Current recording was performed as follow: a ramp protocol between −120 to +80 mV for 2 seconds, holding potential −80 mV, was applied using Clampex (PClamp, Molecular Devices Corporation). To avoid looking at chloride channel activation, current recording was done in MBS where chloride was substituted by gluconate (Na-gluconate 85 mM and K-gluconate 1 mM). Junction potential was minimized using an agar bridge and KCl3M. Electrodes filled with KCl3M were 0.5 MOlun resistance. After equilibration in this gluconate MBS, KCNN4 was activated by the calcium ionophore A23187, 1 μM in MBS gluconate. In control oocytes, no current was activated by ionophore addition.

Western blotting on oocyte: Oocyte membrane were prepared as previously described (Martial et al., 2007). Immunodetection of KCNN4-HA was done using an anti-HA antibody (1/1000, Sigma). To compare KCNN4 expression levels in different samples, the cell membrane marker 0.1 Na,KATPase was used (1/500, Sigma). Signals were detected by chemiluminescent reaction with Immobilon Western reagent (Millipore) and a Fusion FX7 (Vilber-Lourmat, France). The intensity of KCNN4 bands relative to the β1 Na,K-ATPase signal was quantified using ImageJ Version 1.44 software (NCBI).

HEK293 cells transfection: HEK293 cells were grown in DMEM glutamax (Gibco) 10% FBS penicillin-streptomycin. Cells were co-transfected with 1 μg of WT or point mutated pcDNA3-KCNN4-HA and 0.5 μg of pIRES-eYFP using CaPO4. 16 hours later, cells were washed twice with PBS and patch-clamp recordings on fluorescently labeled cells.

Patch-clamp electrophysiology: Glass pipettes (Brand, Wertheim, Germany) were made on a horizontal pipette puller (P-97; Sutter Instrument Co.; Navato, Calif.) to give a final resistance ranging from 3 to 5 MΩ. For whole cell experiments the bath solution was in mM: NaCl 140, KCl5, CaCl₂ 1, Glucose 29, Hepes 25 pH 7.4 adjusted with NaOH. The intracellular solution was in mM: KCl30, KGluconate 100, EGTA 5, Hepes 10 pH 7.2 adjusted with NaOH, CaCl₂ 4.19 (corresponding to 1 μM free calcium), MgATP 2. Currents were measured at room temperature using a ramp protocol form −120 to +80 my from a holding potential of −60 mV (sampling frequency 10 kHz; filtered 1 kHz)

Inside-out recordings: Calcium-dependence of KCNN4 was studied with intracellular (bath) solutions in mM: KCl30, KGluconate 100, EGTA 5, Hepes 10 pH 7.2 adjusted with KOH, CaCl₂ with varying concentrations 4.91; 4.19; 3.61; 2.82; 1.7 (10-5; 10-6; 5.10-7; 2.5.10-7; 10-7 M of free calcium). Maxchelator was used to calculate free Ca²⁺ concentration (http://maxchelator.stanford.edu/CaEGTA-TS.htm). Extracellular solution in mM: NaCl 140, KC1 5, CaCl₂ 1, Glucose 29, Hepes 25 pH 7.4 adjusted with NaOH. Currents were evoked by voltage ramps from −120 to 80 mV (150 ms), filtered at 1 kHz and acquired with a sampling frequency of 10 kHz. All traces were corrected for liquid junction potential. For dose response experiments, normalized values of currents at −45 mV were plotted against free Ca²⁺ concentration.

All patch-clamp experiments were performed with a PC-controlled EPC 9 patch-clamp amplifier (HEKA, Lambrecht/Pfalz, Germany). Currents were acquired and analyzed with Pulse and Pulsefit softwares (HEKA).

Immunohistochemistry: Immunodetection of KCNN4-HA in HEK293 cells was performed using anti-IIA antibody (Sigma-Aldrich).

Red cell cation content and volume measurements: Fresh venous blood was obtained by venipuncture from an informed patient from family 1 and a healthy volunteer. For 24 hours' incubation, blood samples were stored at 37° C. or 4° C.

For vanadate experiments: blood was washed 4 times at room temperature in medium containing (in mM): NaCl (147) KCl (5) MgSO4 (2) CaCl₂ (1) Hepes/NaOH pH7.4 (10). Red cell suspension was then incubated at 37° C., 30% hematocrit and 5 mM vanadate was added alone or with 10 μM TRAM-34. A few minutes before sampling time, 400 μl of cell suspension were taken to fill 3 nylon tubes that were centrifuged for 10 minutes at 4° C., 20000 g at the exact sampling time. The supernatant was collected for extracellular ion content measurements. The pellet of red cells was extracted and immediately weighted wet. Dry weight was measured after overnight heating (80° C.). Water content was calculated with a correction of 3.64% corresponding to trapped medium between packed cells. Intracellular ions were extracted from dried pellets by overnight incubation at 4° C. in 5 ml milliRho water (Millipore). Na⁺ and K⁺ were measured by flame spectroscopy with an Eppendorf ELEX6361.

2. Results

It was initially investigated a fetus (proband 1) for severe in utero anemia without edema, requiring 1 transfusion in utero at week 27 (Hb: 30 g/l). After preterm birth, he received 3 additional transfusions: immediately after birth, at 2 weeks (Hb: 65 g/l) and at 6 weeks of age (Hb: 70 g/l) and was then treated with EPO for 6 weeks. Under treatment, the reticulocyte count progressively increased and Hb value stabilized at 90g/1 at 3 months of age. No further transfusion was necessary. Currently, at 4 years 10 months of age, the proband demonstrated mild anemia and splenomegaly. Clinical history revealed that the mother's proband was affected with a chronic moderate hemolytic anemia of unknown origin from childhood. She was treated with regular transfusion regimen from infancy to adolescence. Chelation therapy was started at 8 years of age and a splenectomy performed at 25 years old. During adult life, she received 2 transfusions, one after a delivery and another one during an infection by the parvovirus B19. Four other members of this family (Family 1) originated from France, were also affected by chronic hemolytic anemia (FIG. 1A). Three out of 4 were splenectomized and 2 of them have received regular transfusions and chelation therapy in periods of time.

In a second unrelated family (Family 2) the proband (proband 2), a 25 year-old person, has suffered from moderate chronic hemolytic anemia since early childhood. She was never transfused and underwent a cholecystectomy because of biliary lithiasis. Her father was originated from Poland and was reported to have severe hemolytic anemia treated by splenectomy and occasional transfusions. Her 2 year-old son was born after a normal pregnancy carried to term, he also presented with a well-tolerated chronic hemolytic anemia. The hematological parameters of proband 1, his mother, proband 2 and her son are summarized in table I below. In addition to anemia, all 4 have a discrete increase of MCHC value.

TABLE 1
Hematological parameters for 4 subjects carrying the
KCNN4 c.1055G > A mutation (representative values
in steady state conditions) values in steady state conditions)

| | | | Reticu- | | | |
|---|---|---|---|---|---|---|
| Normal | | | locytes | | | |
| range | Hb | MCV | count | MCHC | Platelets | Ferritin |
| for | (g/l) | (fl) | (G/l) | (g/l) | (G/l) | (μg/l) |
| adults | 130-160 | 80-100 | 20-80 | 310-350 | 150-400 | 22-322 |
| Proband 1 (age 4) | 98 | 87.9 | 263 | 356 | 319 | 116 |
| Proband 1 mother | 85 | 109 | 255 | 354 | 783 | 94 |
| Proband 2 | 110 | 93.1 | 249 | 361 | 230 | Nd |
| Proband 2's son | 104 | 86.9 | 363 | 365 | 464 | 121 |
| (age 2) | | | | | | |

A microscopic examination of blood smears from proband 1 showed mild anisopoikilocytosis with less than 1% of target cells, polychromatophilic red blood cells, teardrop cells, elliptocytes with sometimes abnormal hemoglobin distribution, hemighosts, bite cells, knizocytes, schizocytes and rare stomatocytic red cells (FIG. 1B). For his mother and for proband 2, anomalies were similar with more significant anisopoikilocytosis and the presence of acanthocytes (FIG. 1B). There was no basophilic stippling of red blood cells.

The EMA test, electrophoresis of red cell membrane proteins and hemoglobin study were normal for all of them. The diagnosis of xerocytosis was not retained initially as there was almost no stomatocyte on blood films and repeated ektacytometry was considered as normal for all 4 tested affected individuals.

Whole exome sequencing was performed for 3 subjects in Family 1, the proband, his affected mother and his unaffected sister. Variants were filtered against dbSNP137 and for heterozygous exonic mutations present in affected individuals only. Thirty-three genes were found carrying exonic, non-synonymous heterozygous mutations among which KCNN4 encoding the Gardos channel, was the most consistent candidate because of its expression in red cells. The missense mutation c.1055G>A (p.Arg352His), confirmed by Sanger sequencing, is located in the Calmodulin interacting region and involves a residue highly conserved among species (FIG. 1C); it was predicted pathogenic by in silico analysis (Polyphen: http://geneties.bwh.harvard.eduipph2), with a score of 0.992 for a maximum of 1. Mutation segregation was studied in 3 other members of Family 1, two affected and one unaffected by chronic hemolysis and was consistent with a dominant transmission of the phenotype linked to the mutation. After direct sequencing of KCNN4 in the 2 affected subjects of Family 2, the same missense mutation c.1055G>A, was identified in heterozygous condition for both of them. Splicing was not affected by the substitution as normal-sized transcripts were heterozygotes for the mutation (FIG. 1D). Using the data of exome sequencing in proband 1, it was confirmed that no mutation was present in FAM38, encoding PIEZOI and described as the major cause of HX up to now.

Further investigations were then performed for proband 1 and his mother. Osmotic fragility was tested to check red cells dehydration. Both mother and son had an abnormal profile after 24 hours of incubation at 37° C.: 50% red cells lysis was obtained with reduced salt concentration when compared to a normal control (FIG. 1E). The profiles were similar to normal control when the same analysis is performed after 24 h at 4° C. explaining why ektacytometry was normal as it was performed after incubation at 4° C. Plasmatic K⁺ concentrations in various conditions of time and temperature after sampling were measured by potentiometry and were in normal ranges. Dynamic efflux of K⁺ under Ca^{21 +} stimulation was assessed by 39K NMA of erythrocytes suspensions using stimulation by the ionophore A23187. Except for a short delay in K⁺ exit following Ca²⁺ activation, no perturbation was observed (data not shown).

In addition, dehydrated red cells are usually associated with haemolytic anemia because shrinkage stimulates Phosphatidylserine (PS) exposure as previously shown in both normal red blood cells, G6PD deficient cells and HbS cells (Lang et at, 2004; Weiss et at, 2011). A relationship between cation leakage and hemolytic anemia has also been observed for other membrane proteins mutations including Band 3 mutations (Bruce et al., 2005). In the present study, it was observed in the patients with mutation in the Gardos channel, a variability in disease severity with a level of anemia rather severe in Family 1 whereas individuals of Family 2 and individuals with PIEZO1 mutations, present normal or subnormal Hb levels (Carella et al., 1998; Houston et al., 2011). Indeed, 2 of the affected individuals from Family 1 exhibit pronounced anemia with an extremely severe episode of in utero anemia for the proband (with no other identified cause, especially no maternal-fetal incompatibility) and numerous transfusions required at many occasions, for his mother. This suggests that susceptibility to scramblase activation resulting from prolonged Ca²⁺ activation and leading to PS exposure may be enhanced in these patients. Iron overload due to chronic anemia is difficult to evaluate in Family I as the mother's proband has regularly been transfused and treated by Deferriprox and the proband himself is too young to suffer from iron overload. In the second family, the patients exhibit moderate iron overload as observed in chronic hemolytic anemia. The function of the Gardos channel variant p.Arg352His was then investigated by the expression of a plasmid containing the mutated cDNA in Xenopus laevis oocytes. The current voltage curves showed that the missense mutation p.Arg352His does not prevent ion conductance through the channel when compared to the wild type channel (FIG. 2A). The activation phase of WT and p.Arg352His channel induced by calcium ionophore was similar but, whereas WT KCNN4 activity decreases after reaching a maximum, the p.Arg352His mutant activity remains quite constant for several minutes. The high and sustained currents with p.Arg352His made it difficult to record for more than 2 minutes after the peak. The reversion potential (−120 mV) was similar between WT and the mutated channel but the current elicited by p.Arg352His KCNN4 expression in Xenopus oocyte was higher than observed with WT (FIG. 2B). Western blots confirmed that both proteins are expressed at similar levels, suggesting that the conductance increase observed for the mutated channel is directly associated with the mutation. These data indicate that the mutation alters the regulation of channel activity favoring a longer activated-state. Inhibition tests performed with TRAM-34, the classical inhibitor of KCNN4, result in decreased current production indicating that the p.Arg352His variant is sensitive to inhibition (FIG. 2C).

To further characterize the p.Arg352His KCNN4, HEK293 cells were transfected with WT or mutated channel. The mutation does not prevent addressing of the channel to plasma membrane. Whole-cell recording shows a different calcium dependent-activation kinetic for HEK293 cells expressing WT or p.Arg352His KCNN4 (FIG. 3A). For the former, the current progressively appears while Ca²⁺ diffuses from the pipette to the intracellular compartment. By contrast, in the latter case, the current is activated immediately after break-in for the mutant and further increases during the time of recording. As in oocyte experiments, the maximum current density is increased in p.Arg352His KCNN4 expressing cells (FIG. 3A-C). These results suggest that the mutation increases channel sensitivity to calcium. The leftward shift in reversal potential observed between break-in and steady-state for WT confirms the delay due to Ca²⁺ diffusion to activate the channel. This delay is not observed for p.Arg352His KCNN4. The calcium sensitivity of WT versus mutated KCNN4 was further explored by performing giant excised inside-out patch-clamp experiments. FIG. 3D shows representative traces of K⁺ currents as a function of voltage and Ca²⁺ concentrations applied to the internal face of the membrane. In FIG. 3E, currents at −45 mV are plotted as a function of Ca²⁺ concentrations. Quantitative analysis showed the calcium dependence of the WT KCNN4 to have an apparent Kd of 0.95 μM±0.09 whereas the apparent Kd is 0.21 μM±0.02 for p.Arg352His mutant. The Hill coefficients are not statistically different between WT and mutant KCNN4 (3.75±1.45 and 3.3±0.85 respectively, n=4).

According to electrophysiological data, KCNN4 should be activated by lower calcium concentration in patient red cells compared to control. To assess the effect of an increase in intracellular Ca²⁺ on the kinetic of Gardos channel activation in control or patient red cells, the net potassium flux was measured in red cells treated by vanadate. Vanadate increases intracellular Ca²⁺ concentration in red cells by inhibiting the calcium pump and also by activating the calcium influx (Varecka and Carafoli, 1982; Bennekou et al., 2012). FIG. 4 illustrates the K⁺content of control or patient red cells in presence of 5 mM vanadate with or without 10 μM TRAM-34. Whereas vanadate did not significantly change intracellular K⁺ content in control red cells in 1 hour, a significant decrease in intracellular K⁺ was observed in patient red cells and this decrease was blocked by TRAM-34. The K⁺ efflux is correlated to cell volume decrease as illustrated on FIG. 4B. No significant change in Na⁺ contents was observed in control and in patient red cells at the same time (FIG. 4C).

The K⁺ content of red cells in blood stored for 24 hours at 37° C. or 4° C. is given in table 2 below.

TABLE 2
K⁺ content in red cells as a function of blood temperature. Data are
expressed in μmol per gram of dry weight
(μmol/g d.w. +/− S.D. for 3 samples).

| K⁺ content | t0 | 24 h 37° C. | 24 h 4° C. |
|---|---|---|---|
| Control | 281.1 +/− 4.9 | 260.0 +/− 1.7 | 257.0 +/− 3.4 |
| Proband 1′aunt | 316.9 +/− 5.5 | 223.1 +/− 10.2 | 281.6 +/− 4.6 |
| (affected) | | | |

In control red cells, the K⁺content is decreased by 21.1 μmol/g d.w. after 24 h at 37° C. This variation is similar for blood stored for 24 h at 4° C. (−24.1 μmol/g d.w.). In contrast, there is a K⁺ loss of 93.8 μmol/g d.w. in patient red cells stored at 37° C. compared to 35.3 μmol/g d.w. for patient blood stored for 24 h at 4° C.

### EXAMPLE II

### Use of Senicapoce for the Treatment of a Subset of Hereditary Xerocytosis Caused by Mutations in the Gardos Channel

1. Material and Methods

Plasmid pcDNA3-KCNN4-HA was used to introduce the point mutation V282M and V282E by PCR as described above.

HEK293 cells transfection: HEK293 cells were grown in DMEM glutamax (Gibco) 10% FBS 1% penicillin-streptomycin. Cells were co-transfected with 1 μg of wilt-type (WT) or point mutated pcDNA3-KCNN4-HA and 0.5 μg of pIRES-eYFP using CaPO₄. 16 hours later, cells were washed twice with PBS and patch-clamp was performed on fluorescence-labeled cells.

Patch-clamp electrophysiology: Glass pipettes (Brand, Wertheim, Germany) were made on a horizontal pipette puller (P-97; Sutter Instrument Co.; Navato, Calif.) to give a final resistance ranging from 3 to 5 MΩ. For whole-cell experiments in HEK cells, the bath solution was in mM: NaCl 145, KCl 5, CaCl₂ 2, MgCl₂ 1, Hepes 10 pH 7.4 adjusted with NaOH (320 mOsm). The intracellular solution was in mM: KCl 145, MgCl₂1, Hepes 10, pH 7.2 adjusted with KOH, CaCl₂ 0.87 EGTA I (corresponding to 1 μM free calcium. Maxchelator software was used to calculate free Ca²⁺ concentration (http://maxchelator.stanford.edu/CaEGTA-TS.htm)) (305 mOsm). Currents were measured at room temperature using a ramp protocol from −120 to +80 mV from a holding potential of −60 mV (sampling frequency 10 kHz; filtered 1 kHz).

For whole-cell experiments in human red blood cell, glass pipettes (Brand, Wertheim, Germany) were made on a vertical pipette puller (PIPS; HEKA, Lambrecht/Pfalz, Germany) to give a final resistance ranging from 17 to 20 MΩ. The same solution was used for pipette and bath and contained in mM: KCl 150, NaCl 5, MgCl₂ 1, Hepes 10, CaCl₂1, pH 7.4 (320 mOsm). Currents were measured at room temperature using a ramp protocol from −40 to +70 mV during 800 ms from a holding potential of −20 mV (sampling frequency 10 kHz; filtered 1 kHz). All patch-clamp experiments were performed with a PC-controlled EPC 9 patch-clamp amplifier (HEKA, Lambrecht/Pfalz, Germany). Currents were acquired and analyzed with Pulse and Pulsefit softwares (HEKA).

Hematological tests: Fresh venous blood was obtained by venipuncture from an informed patient from Family I (see above), and healthy volunteers. An osmotic fragility test in hypotonic saline solutions, was performed on red blood cells after 25 hours' incubation at 4° or 37° C. in presence or absence of 10 μM TRAM-34 or Senicapoc at 0.4 or 4 μM.

Red blood cell cation content and volume measurements: Freshly drawn blood was washed 4 times at room temperature in medium containing in mM: NaCl 147, KCl 5, MgSO4 2, CaCl₂ 1, Hepes 10, buffered with NaOH pH 7.4 (320 mOsm). Red blood cell suspension was then incubated at 37° C., 30% hematocrit and 5 mM vanadate was added alone or with 10 μM TRAM-34 or different concentrations of Senicapoc. A few minutes before sampling time, 400 μl of cell suspension was taken to fill 3 nylon tubes that were centrifuged for 10 minutes at 4° C., 20,000. g at the exact sampling time. The supernatant was collected for extracellular ion content measurements. The pellet of red cells was extracted and immediately weighted. Then, dry weight was measured after overnight heating (80° C.). Water content was calculated with a correction of 3.64% corresponding to trapped medium between packed cells. Intracellular ions were extracted from dried pellets by overnight incubation at 4° C. in 5 ml milliRho water (Millipore). Na⁺ and K⁺ were measured by flame spectroscopy with a PFP7 Jenway. Statistics: Mann and Whitney test was used to compare control versus patient or control versus inhibitor in red blood cell experiments.

2. Results

In order to study and compare the different mutations of KCNN4 linked to HX, HEK293 cells were used as a reliable heterologous expression system that allowed to overcome the difficulties to do patch-clamp on HX red blood cells.

Current Features of KCNN4 Mutants V282M and V282E

HEK293 cells were transiently transfected with WT KCNN4 or the two mutants on Val282, V282E and V282M, and currents were then recorded in whole cell configuration. FIG. 5A shows that the two substitutions on Val282 increased current density compared to WT. To assess the calcium sensitivity of these currents, the same experiment was done with two different intracellular Ca²⁺ concentrations: 1 μM, corresponding to the EC50 for WT KCNN4 and 0.25 μM corresponding to the EC50 of R352H mutant (see above). FIG. 5B-C shows that there is a similar activity of V282E and WT KCNN4 at the two intracellular calcium concentrations. However, this calcium sensitivity is not observed for V282M mutant, which has a similar current density for 0.25 and 1 μM of intracellular Ca²⁺ (FIG. 5D).

Sensitivity to Senicapoc

Senicapoc sensitivity of the 3 different mutations linked to HX was assessed on HEK cells transiently transfected with each construct. FIG. 6A shows representative current/voltage curves for WT and mutated KCNN4 as a function of different concentrations of Senicapoc. It was observed that V282E is almost insensitive to Senicapoc, only a slight inhibition being observed for 10 μM Senicapoc. By contrast, R352H mutant is much more sensitive to Senicapoc than the WT channel. WT and V282M channels exhibit a similar Senicapoc sensitivity. FIG. 6B summarizes as dose-response curves data of inhibition between WT and the two mutants R352H and V282M. The IC50 around 10 nM is not statistically different for WT and V282M. By contrast, the R352H mutant is about 30 times more sensitive than the WT with an IC50=0.3 nM (FIG. 6C).

Senicapoc Effects on Red Cells with a R352H Mutation

Fresh blood samples were obtained from a patient carrying the R352H mutation on Gardos channel. To assess whether the inhibitor could be efficient on mutated Gardos channel in red blood cells as in HEK293 cell, its effect was evaluated on 1) the K⁺ loss following Gardos channel activation, 2) red blood cell osmotic resistance and 3) red blood cell Ca²⁺ activated K⁺ current. Senicapoc was used at higher concentrations than in HEK293 cells to account for the high hemoglobin concentrations in experiments with blood.

1) Red Blood Cell K⁺ Loss

The Gardos channel was activated by intracellular Ca²⁺ increase. Vanadate was used to block Ca²⁺ pump and increase intracellular Ca²⁺ as described previously (Bennekou et al., 2012; Rapetti-Mauss et al., 2015). FIG. 7 illustrates the kinetic of K⁺ loss induced by 5 mM vanadate in control (A) and patient (B) red blood cells. This K⁴ loss was correlated to cell volume decrease (FIG. 7C-D) and there was no change in intracellular Na⁺ content (not shown). This figure shows that both, TRAM-34 and Senicapoc, were able to reduce K⁴ loss and cell water loss in red blood cells with WT or R352H Gardos channels.

After one hour incubation with vanadate, a large K⁺ efflux was observed in patient red blood cells compared to control; −92±14 mol/g d.w. versus −53±16 mol/g d.w., means±sem of 3 independent experiments (significant difference with p<0.05). At 60 minutes, there is a 97±1% inhibition of K⁺ loss by 10 μM TRAM-34 in control red blood cells and a 92±3% inhibition in patient red blood cells (means±sem of 3 independent experiments, p<0.05 control versus TRAM-34 in both patient and control red blood cells). Senicapoc at 0.4 μM inhibited K⁺ loss by 79±12% and 84±4% for patient and control red blood cells respectively (p<0.05 control versus senicapoc in both patient and control red blood cells; non significant for control versus patient red blood cells). A 10 times higher Senicapoc concentration (4 μM) was assessed on a single time point (60 min.). For 4 μM Senicapoc, K⁺ loss after 60 minutes with vanadate was inhibited by 95±4% and 94±9% in control and patient red blood cells respectively (p<0.05 control versus senicapoc in both patient and control red blood cells).

2) Osmotic Resistance

Freshly drawn blood was stored for 25 hours at 37° C. or 4° C. in presence of TRAM-34 (10 μM) or Senicapoc (4 or 0.4 μM) and compared to control condition. The osmotic resistance is similar between patient and control for blood incubated at 4° C. and Senicapoc has no effect (FIG. 8A-B). By contrast, the osmotic resistance of patient red blood cells at 37° C. is shifted to the left compared to control red blood cells, giving 50% hemolysis of patient and control red blood cells, respectively at 0.45 and 0.5 relative osmolarity (FIG. 8C-D black diamond curves). The slope of the curve is also dramatically reduced for patient compared to control blood. Whereas incubation with Senicapoc did not alter osmotic resistance curve of control blood (FIG. 8C), there is a dose dependent effect of Senicapoc only on patient red blood cells. In these latter, the osmotic resistance is shifted to the right and the steepness of the curves increases in presence of Senicapoc (FIG. 8D triangles and crosses). TRAM-34 is able to decrease the osmotic resistance in control and patient red blood cells, very slightly at 4° C. and more significantly at 37° C. It was observed that before the test, TRAM-34 induced a strong hemolysis in patient as well as in control red blood cells. This was not observed with Senicapoc.

3) Red Blood Cell Native Current

FIG. 9 illustrates the activity of Gardos channels recorded in patient (R352H mutation) and control red blood cells in whole-cell configuration. A significant current increase is observed for patient red blood cells compared to control. Both currents are completely blocked by 0.5 μM Senicapoc or 10 μM TRAM-34.

### REFERENCES

### Andolfo I, et al., 2013a, Blood. 121:3925-3935.

### Andolfo I, et al., 2013b, Am J Hematol, 88:66-72.

### Andolfo I, et al., 2015, Am J Hematol. 90:921-926.

### Archer N M, et al., 2014, Am J Hematol. 89:1142-1146.

### Ataga K I, et al., 2008, Blood. 111:3991-3997.

### Ataga K I, et at., 2009, Expert Opin Investig Drugs. 18:231-239.

### Ataga K I, et al., 2011, Br J Haematol. 153:92-104.

### Bae C, et al., 2013, Proc Natl Acad Sci U S A. 110:E1162-1168.

Barneaud-Rocca et al., 2011, J Biol. Chem. 286:8909-8916.
Bennekou P, et al., 2012. Blood Cells Mol. Dis. 48:102-109.

### Bruce L J, et al., 2005, Nat Genet. 37:1258-1263.

### Brugnara C, et al., 1993a, J Clin Invest. 92:520-526.

Brugnara C, et al., 1993b, J. Biol. Chem. 268:8760-8768

### Brugnara C, et al., 1996, J Clin Invest. 97:1227-1234.

### Carella M, et al., 1998, Am J Hum Genet. 63:810-816.

Castle A, et al., 2002, Mal. Pharmacol. 63:409-18.

### Da Costa L, et al., 2013, Blood Rev. 27:167-178.

Dyrda A, et al., 2010, PLoS One. 5:e9447.

### Ellory J C, et al., 1994, Br J Pharmacol. 111:903-905

### Fanger C M, et al., 1999, J Biol Chem. 274:5746-5754.

### Glogowska E, et al., 2015, Blood. 126:1281-1284.

### Houston B L, et al., 2011, Blood Cells Mol Dis. 47:226-231.

### Joiner W J, et al., 1997, Proc Natl Acad Sci U S A. 94:11013-11018.

### Joiner W J, et al., 2001, J Biol Chem. 276:37980-5.

Kaplan J M and Delpech M., 2007, Biologie moléculaire et médecine (3° Éd.) (Coll. De la biologic a la clinique), Ed. Flammarion

### Lang F, et al., 2004, Adv Exp Med Biol. 559:211-217.

### Maher A D, Kuchel P W, 2003, Int J Biochem Cell Biol. 35:1182-1197.

### Martial S, et al., 2007, J Cell Physiol. 213:70-78.

### Miller D R, et al., 1971, Blood. 38:184-204.

### Morales P, et al., 2013, J Gen Physiol. 142:37-60.

### Quinlan A R and Hall I M, 2010, Bioinformatics. 26:841-842.

### Rapetti-Mauss R, et al., 2015, Blood. 126:1273-1280.

### Rinehart J, et al., 2010, Curr Opin Hematol. 17:191-197.

### Romero JR, et al., 2002, Blood. 99:1103-1108.

### Stocker W, et al., 2003, Blood. 101:2412-2418.

### Varecka L and Carafoli E., 1982, J Biol Chem. 257:7414-7421.

Wang K, et al., 2010, Nucleic Acids Res.; 38: e164.

### Weiss E, et al., 2011, Anemia. 2011:379894.

### Wulff H, Castle N A, 2010, Expert Rev Clin Pharmacol. 3:385-396.

### Wulff H, et al., 2000, PNAS. 97:8151-8156.

### Wulff H, Köhler R, 2013, J Cardiovasc Pharmacol. 61:102-112.

### Zarychanski R, et al., 2012, Blood. 120:1908-1915.

1. An inhibitor of the Gardos channel (KCNN4 protein) for use in the treatment of hereditary xerocytosis, wherein said inhibitor is a selective Gardos channel blocker that specifically inhibits the efflux of potassium from the erythrocytes.

2. The inhibitor for use according to claim 1, wherein the inhibitor is selected from the group consisting of an organic molecule, an amino acid and an antibody.

3. The inhibitor for use according to claim 2, wherein the inhibitor is selected from the group consisting of imidazole antimycotics, clotrimazole, metronidazole, econazole, arginine, Tram-34, harybdotoxin, nifedipine, 2,2-Bis(4-fluorophenyl)-N-methoxy-2-phenylacetamidine, 2-(2-Chlorophenyl)-2,2-diphenylacetaldehyde oxime, 2-(2-Chlorophenyl)-2,2-bis(4-fluorophenyl)-N-hydroxyacetamidine, 2,2,2-Tris(4-fluorophenyl)-N-hydroxyacetamidine, 2-(2-Fluorophenyl)-2-(4-fluorophenyl)-N-hydroxy-2-phenylacetamidine, phosphoric acid 3-(2-oxazolyl)-4-[3-(trifluoromethyl)phenylsulfonamido]phenyl monoester, N-[2-(4,5-Dihydrooxazol-2-yl)phenyl]-3-(trifluoromethyl)benzenesulfonamide, N-[4-Methoxy-2-(2-oxazolyl)phenyl]benzenesulfonamide, N-[4,5-Dimethoxy-2-(3-methyl-1,2,4-oxadiazol-5-yl)phenyl]-3-(trifluoromethyl)benzenesulfonamide, N-[2-(2-Furyl)phenyl]-3-(trifluoromethyl)benzenesulfonamide and N-[4-Methyl-2-(2-oxazolyl)phenyl]-3-(trifluoromethyl)benzenesulfonamide and senicapoc, preferably senicapoc.

4. The inhibitor for use according to claim 1, wherein the inhibitor is used in the treatment of hereditary xerocytosis of a human subject who is a carrier of a missense mutation selected from the group consisting of c.1055G>A, c.844G>A or c.845T>A, in the KCNN4 gene encoding the Gardos channel, resulting respectively in an amino acid change from arginine to histidine in codon 352, in an amino acid change from valine to methionine in codon 282 or in an amino acid change from valine to glutamine in codon 282, preferably c.1055G>A or c.844G>A, preferably c.1055G>A.

5. A method for genotyping, in vitro, the KCNN4 gene in a human subject comprising the steps of:
(a) isolating mRNA or genomic DNA from a nucleic acid sample obtained from said subject,
(b) determining the nucleotide present at position c.1055 of the KCNN4 gene encoding the Gardos channel.

6. The method according to claim 5, wherein the mRNA or genomic DNA is obtained from a blood sample from said subject.

7. The method according to claim 5 wherein the mRNA is obtained from reticulocytes from said subject and the genomic DNA is obtained from white blood cells from said subject.

8. The method according to claim 5, wherein said step (b) is carried out by hybridization techniques, selective amplification, nucleic acid sequencing, restriction fragment length polymorphism (RFLP), amplified fragment length polymorphism (AFLP), ligation chain reaction (LCR) or mass spectrometry.

9. The method according to claim 1, wherein said the nucleotide at position c.1055 is determined by reverse dot blot using the probes of SEQ ID NO: 3 and SEQ ID NO: 4.

10. An in vitro method of diagnosing the presence of or predisposition to hereditary xerocytosis in a human subject, comprising the step of:
(i) providing a biological sample from said subject and
(ii) detecting in said biological sample the presence of the missense mutation c.1055G>A in the KCNN4 gene encoding the Gardos channel or the missense mutation p.Arg352His in the Gardos channel (KCNN4 protein),
the presence of said mutation constituting a marker of a hereditary xerocytosis or a predisposition to hereditary xerocytosis in said subject.

11. The method according to claim 10, wherein the presence of the missense mutation c.1055G>A in the KCNN4 gene encoding the Gardos channel is detected by genotyping the KCNN4 gene in said human subject according to the method of any one of claims 5 to 9.

12. The method according to claim 10, wherein the presence of the mutation p.Arg352His in the Gardos channel is detected by protein sequencing or binding to a ligand specifically directed to the Gardos channel variant p.Arg352His.

13. A kit for diagnosing a hereditary xerocytosis comprising the probe of SEQ ID NO: 3 and/or the probe of SEQ ID NO: 4.

14. Use of the probe of SEQ ID NO: 3 and/or the probe of SEQ ID NO: 4 for in vitro diagnosing a hereditary xerocytosis in a human subject.

15. An in vitro a method for screening a biologically active inhibitor of the human Gardos channel (KCNN4 protein) variant selected from the group consisting of p.Arg352His, p.Val282Met or p.Val282Glu, said method comprising contacting in vitro a test compound with the human Gardos channel (KCNN4 protein) variant p.Arg352His, p.Val282Met or p.Val282Glu, respectively and determining the ability of said test compound to prevent ion conductance through the channel when compared to the wild-type human Gardos channel (KCNN4 protein) of SEQ ID NO: 2, wherein preventing ion conductance through the channel when compared to the wild-type human Gardos channel (KCNN4 protein) of SEQ ID NO: 2 provides an indication as to the ability of the compound to inhibit the human Gardos channel (KCNN4 protein) variant p.Arg352His, p.Val282Met or p.Val282Glu.


SEQUENCE LISTING (14 sequences)

SEQ ID NO: 1 - DNA, 2240 bp, Homo sapiens
  CDS (397)..(1680)
gtccttcggtgtctgggtgtggtgagtagaggtgtgtgtcacaaagtacagaccattgtgtgtgacaaagcccatcgtgtgtctgtgtgtgtctttatccacgtggatggacgtctctttcttgctctgccccaagacacaccctagcccctccttattctcaaaagggggagctggggagcctccccctaccctggggcctcccctgcccctccccgccctgcctggccgtcaccactccccagagggcacagggctctgctgtgcctcagagcaaaagtcccagagccagcagagcaggctgacgacctgcaagccacagtggctgccctgtgcgtgctgcgaggtgggggaccctgggcaggaagctggctgagccccaagaccccgggggccatgggcggggatctggtgcttggcctgggggccttgagacgccgaaagcgcttgctggagcaggagaagtctctggccggctgggcactggtgctggcaggaactggcattggactcatggtgctgcatgcagagatgctgtggttcggggggtgctcgtgggcgctctacctgttcctggttaaatgcacgatcagcatttccaccttcttactcctctgcctcatcgtggcctttcatgccaaagaggtccagctgttcatgaccgacaacgggctgcgggactggcgcgtggcgctgaccgggcggcaggcggcgcagatcgtgctggagctggtggtgtgtgggctgcacccggcgcccgtgcggggcccgccgtgcgtgcaggatttaggggcgccgctgacctccccgcagccctggccgggattcctgggccaaggggaagcgctgctgtccctggccatgctgctgcgtctctacctggtgccccgcgccgtgctcctgcgcagcggcgtcctgctcaacgcttcctaccgcagcatcggcgctctcaatcaagtccgcttccgccactggttcgtggccaagctttacatgaacacgcaccctggccgcctgctgctcggcctcacgcttggcctctggctgaccaccgcctgggtgctgtccgtggccgagaggcaggctgttaatgccactgggcacctttcagacacactttggctgatccccatcacattcctgaccatcggctatggtgacgtggtgccgggcaccatgtggggcaagatcgtctgcctgtgcactggagtcatgggtgtctgctgcacagccctgctggtggccgtggtggcccggaagctggagtttaacaaggcagagaagcacgtgcacaacttcatgatggatatccagtataccaaagagatgaaggagtccgctgcccgagtgctacaagaagcctggatgttctacaaacatactcgcaggaaggagtctcatgctgcccgcaggcatcagcgcaagctgctggccgccatcaacgcgttccgccaggtgcggctgaaacaccggaagctccgggaacaagtgaactccatggtggacatctccaagatgcacatgatcctgtatgacctgcagcagaatctgagcagctcacaccgggccctggagaaacagattgacacgctggcggggaagctggatgccctgactgagctgcttagcactgccctggggccgaggcagcttccagaacccagccagcagtccaagtagctggacccacgaggaggaaccaggctactttccccagtactgaggtggtggacatcgtctctgccactcctgacccagccctgaacaaagcacctcaagtgcaaggaccaaagggggccctggcttggagtgggttggcttgctgatggctgctggaggggacgctggctaaagtgggtaggccttggcccacctgaggccccaggtgggaacatggtcacccccactctgcataccctcatcaaaaacactctcactatgctgctatggacgacctccagctctcagttacaagtgcaggcgactggaggcaggactcctgggtccctgggaaagagggtactaggggcccggatccaggattctgggaggcttcagttaccgctggccgagctgaagaactgggtatgaggctggggcggggctggaggtggcgccccctggtgggacaacaaagaggacaccatttttccagagctgcagagagcacctggtggggaggaagaagtgtaactcaccagcctctgctcttatctttgtaataaatgttaaagccagaa

SEQ ID NO: 2 - protein, 427 aa, Homo sapiens
MGGDLVLGLGALRRRKRLLEQEKSLAGWALVLAGTGIGLMVLHAEMLWFGGCSWALYLFLVKCTISISTFLLLCLIVAFHAKEVQLFMTDNGLRDWRVALTGRQAAQIVLELVVCGLHPAPVRGPPCVQDLGAPLTSPQPWPGFLGQGEALLSLAMLLRLYLVPRAVLLRSGVLLNASYRSIGALNQVRFRHWFVAKLYMNTHPGRLLLGLTLGLWLTTAWVLSVAERQAVNATGHLSDTLWLIPITFLTIGYGDVVPGTMWGKIVCLCTGVMGVCCTALLVAVVARKLEFNKAEKHVHNFMMDIQYTKEMKESAARVLQEAWMFYKHTRRKESHAARRHQRKLLAAINAFRQVRLKHRKLREQVNSMVDISKMHMILYDLQQNLSSSHRALEKQIDTLAGKLDALTELLSTALGPRQLPEPSQQSK

SEQ ID NO: 3 - DNA, 25 bp, Artificial Sequence
  Probe
cccacaggttccgccaggtgcggct

SEQ ID NO: 4 - DNA, 25 bp, Artificial Sequence
  Probe
cccacaggttccaccaggtgcggct

SEQ ID NO: 5 - DNA, 20 bp, Artificial Sequence
  Primer forward
agtgctacaagaagcctgga

SEQ ID NO: 6 - DNA, 20 bp, Artificial Sequence
  Primer reverse
tgctaagcagctcagtcagg

SEQ ID NO: 7 - protein, 22 aa, Homo sapiens
KLLAAINAFRQVRLKHRKLREQ

SEQ ID NO: 8 - protein, 21 aa, Pan troglodytes
KLLAAINAFRQVRLKHRKLRE

SEQ ID NO: 9 - protein, 16 aa, Macaca mulatta
INAFRQVRLKHRKLQE

SEQ ID NO: 10 - protein, 18 aa, Felis catus
RLLAAINRFRQVRLKHRK

SEQ ID NO: 11 - protein, 18 aa, Mus musculus
KMLAAIHTFRQVRLKHRK

SEQ ID NO: 12 - protein, 21 aa, Drosophila melanogaster
KFLLAIYALRKVKMDQRKLMD

SEQ ID NO: 13 - protein, 16 aa, Caenorhabditis elegans
KFLLAIYEMRRVRRDQ

SEQ ID NO: 14 - protein, 21 aa, Xenopus tropicalis
NLLRAIHVFRRSRISHKNLKD